(19) United States
(12) Patent Application Publication
(10) Pub. No.: US 2025/0179521 A1
(43) Pub. Date: Jun. 5, 2025

(54) Spatially Localized Gene Delivery of Adeno-Associated Viruses

(71) Applicant: Massachusetts Institute of Technology, Cambridge, MA (US)

(72) Inventors: Polina Olegovna ANIKEEVA, Lexington, MA (US); Keisuke Nagao, Somerville, MA (US)

(73) Assignee: Massachusetts Institute of Technology, Cambridge, MA (US)

(21) Appl. No.: 18/903,558

(22) Filed: Oct. 1, 2024

Related U.S. Application Data
(60) Provisional application No. 63/605,120, filed on Dec. 1, 2023.

(51) Int. Cl. C12N 15/86; C12N 15/87

(52) U.S. Cl. CPC C12N 15/86; C12N 15/87; C12N 2750/14143

(57) ABSTRACT

A gene-targeting chimera including an adeno-associated virus (AAV) and a nanoparticle is disclosed. The nanoparticle may include a silica shell coating. The AAV and nanoparticle may be covalently attached using a linking chemistry. The nanoparticle may include a magnetic nanoparticle (MNP), a magnetic nanodisc (MND), or a quantum dot (QD). The gene-targeting chimeras may retain the tropism of original AAV serotype used. Additionally, The gene-targeting chimeras may also be able to be controlled using a magnetic field. The gene-targeting chimeras may enable nanoparticle delivery to specific cells and organs.

### CROSS REFERENCE TO RELATED APPLICATION(S)

The present application claims the priority benefit, under 35 U.S.C. 119 (e), of U.S. Application No. 63/605,120, filed Dec. 1, 2023, which is incorporated herein by reference in its entirety for all purposes.

### GOVERNMENT SUPPORT

This invention was made with government support under AT011991 awarded by the National Institutes of Health. The government has certain rights in the invention.

### BACKGROUND

The delivery of therapeutics across the blood-brain barrier (BBB) remains a critical challenge to the effective treatment of neurological diseases. Although numerous strategies, including grafting poly(ethylene glycol) (PEG) and targeting moieties (e.g., antibodies, peptides) onto cargo and employing extracellular vesicles, have been explored, the delivery efficiency into the brain may remain approximately ≤1% of the injected dose (ID) and significant off-target accumulation is commonly observed in the liver. Consequently, fundamentally different approaches are needed to accelerate efficient and targeted delivery of therapeutics into the brain.

In nature, viruses have evolved to effectively target specific tissues, and this functionality is extensively exploited in the fields of gene delivery and therapy. In particular, adeno-associated viruses (AAVs) possess low immunogenicity and non-pathogenicity, making them preferred gene delivery vectors. AAV capsids include 60 protein units self-assembled into an icosahedral structure that endows them with the ability to target specific tissues while preventing non-specific interactions with the others. Vigorous research into AAV tropism have delivered serotypes that effectively target a variety of cell classes, including a recently reported AAV.CAP-B10 that efficiently delivers transgenes to the brain while minimizing transduction in the liver, which disproportionally accumulates and filters nanoscale objects such as AAVs from the blood.

### SUMMARY

In some aspects, the techniques described herein relate to a gene-targeting chimera including at least one adeno-associated virus (AAV), a nanoparticle, a uniform silica shell coating over the nanoparticle, and a linking chemistry covalently attaching the at least one AAV and the nanoparticle.

In some aspects, the techniques described herein relate to a gene-targeting chimera, wherein the at least one AAV is up to eight AAVs.

In some aspects, the techniques described herein relate to a gene-targeting chimera, wherein the at least one AAV consists of one AAV.

In some aspects, the techniques described herein relate to a gene-targeting chimera, wherein the nanoparticle includes at least one of a magnetic nanoparticle (MNP), a magnetic nanodisc (MND), or a quantum dot (QD).

In some aspects, the techniques described herein relate to a gene-targeting chimera, wherein the nanoparticle is the MNP.

In some aspects, the techniques described herein relate to a gene-targeting chimera, wherein the MNP is about 20 nm to about 25 nm in diameter.

In some aspects, the techniques described herein relate to a gene-targeting chimera, wherein the MNP includes a spherical MNP or a faceted MNP.

In some aspects, the techniques described herein relate to a gene-targeting chimera, wherein the linking chemistry includes a click chemistry reaction.

In some aspects, the techniques described herein relate to a gene-targeting chimera, wherein the click chemistry reaction is an inverse electron-demand dials-alder (IEDDA) reaction.

In some aspects, the techniques described herein relate to a gene-targeting chimera, wherein the uniform silica shell coating is about 2 nm to about 8 nm thick.

In some aspects, the techniques described herein relate to a gene-targeting chimera, wherein a location of the gene-targeting chimera in a mammal may be controlled by at least one of a serotype of the AAV or a magnetic field.

In some aspects, the techniques described herein relate to a gene-targeting chimera, wherein the gene-targeting chimera is contained in a solution including a plurality of AAVs and nanoparticles wherein each AAV is covalently attached to the nanoparticle.

In some aspects, the techniques described herein relate to a method including coating a nanoparticle with a uniform shell of silica, covalently attaching the nanoparticle to at least one adeno-associated virus (AAV) to form a gene-targeting chimera, wherein the at least one AAV is up to eight AAVs, and intravenously injecting the gene-targeting chimera into a mammal.

In some aspects, the techniques described herein relate to a method, further including controlling a location of the gene-targeting chimera using at least one of a magnetic field or a serotype of the AAV within the mammal.

In some aspects, the techniques described herein relate to a method, wherein the covalently attaching the nanoparticle to the AAV further includes controlling a ratio of the at least one AAV to the nanoparticle using a quenching agent.

In some aspects, the techniques described herein relate to a method, wherein the quenching agent includes tetrazine-methoxy polyethylene glycol or trans-cyclooctene methoxy polyethylene glycol.

In some aspects, the techniques described herein relate to a method, wherein the covalently attaching the nanoparticle to the AAV includes a click chemistry reaction.

In some aspects, the techniques described herein relate to a method, wherein the click chemistry reaction includes an inverse electron-demand dials-alder (IEDDA) reaction.

In some aspects, the techniques described herein relate to a method, wherein the coating the nanoparticle with silica includes adding oleic acid to control a thickness of the uniform shell of silica.

In some aspects, the techniques described herein relate to a method, wherein the nanoparticle includes at least one of a magnetic nanoparticle (MNP), a magnetic nanodisc (MND), or a quantum dot (QD).

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A illustrates an organ specific systemic delivery with various gene targeting chimeras including a magnetic nanoparticle (MNP)-AAV chimera and a quantum dot (QD) chimera.

FIG. 1B illustrates a visualization of accessible lysine residues on an AAV9 capsid.

FIG. 1C illustrates a reaction scheme depicting functionalization of AAVs and MNPs and subsequent chimerization.

FIG. 1D illustrates the reaction scheme depicting functionalization of AAVs and MNPs and subsequent chimerization.

FIG. 1E is a transmission electron microscope (TEM) image showing a Tz-functionalized AAV at stage i of the synthesis shown in FIG. 1D. Scale bar, 50 nm.

FIG. 1F is a TEM image showing an as-synthesized MNP at stage ii of the synthesis shown in FIG. 1D. Scale bar, 50 nm.

FIG. 1G is a TEM image showing a silica-coated amine-functionalized MNP at stage iii of the synthesis shown in FIG. 1D. Scale bar, 50 nm.

FIG. 1H is a TEM image showing a silica-coated TCO-functionalized MNP at stage iv of the synthesis shown in FIG. 1D. Scale bar, 50 nm.

FIG. 1I is a TEM image showing an MNP-AAV chimera at stage v of the synthesis shown in FIG. 1D. Scale bar, 50 nm.

FIG. 1J is a graph showing Zeta potential measurements of MNP@SiO₂—NH₂ and MNP@SiO₂-TCO.

FIG. 2A illustrates TEM images of MNP-AAV chimeras with different \dot{χ} from 1 to 7. Scale bars, 20 nm.

FIG. 2B illustrates a TEM image of a QD chimera of χ=1. Scale bar, 20 nm.

FIG. 2C illustrates representative TEM images of MNP-AAV chimeras made of AAV.CAP-B10, AAV9, and AAV-DJ with the same χ̅. Scale bars, 100 nm.

FIG. 2D is a graph showing the relationship between the AAV/MNP ratio during the chimerization reaction and the resulting χ̅ of product chimeras. Approximately >600 objects were used to evaluate χ̅.

FIG. 3A is confocal microscope images of HEK293T cells incubated with (left) AAV-DJ chimeras, (middle) AAV9 chimeras, and (right) AAV-free MNP control for 4 hours at 0.05 μg-Fe/mL of chimeras or MNPs. The cytoplasm was stained with CellTracker™ Green before being fixed. The nucleus was stained with DAPI. Scale bars, 10 μm.

FIG. 3B is a graph showing the average intensity of Cy7 fluorescence per cell for each sample shown in FIG. 3A (Welch's t-test. n=600. *** P≤0.001).

FIG. 3C is a scatter plot of the percentage of Cy7⁺ cells (MNP-containing) versus the percentage of mRuby2⁺ cells (transduced by an AAV) for each combination of an AAV serotype and a cell line. The Pearson's correlation coefficient (r) was 0.839. The AAV serotype is included in accompanying Table 2.

FIG. 4A illustrates live cell imaging of HEK293T cells incubated with AAV-DJ chimeras. Cells were imaged right before chimeras were added (−0 min, first panel) and 10 minutes (second panel) and 2 hours (third panel) post chimera addition. The top panel of images are zoom-ins of the cell membrane indicated by rectangles in the lower panel of images. The fourth panel box at t=2 hours shows a zoom-in image of the perinuclear region indicated in the third panel with the top square box. Blue-DAPI, Green-CellMask™ plasma membrane dye, Magenta-Cy7 attached to MNPs. CellMask dye stained membrane-associated lipids, which also visualized some intracellular compartments as well as the cell membrane. HEK293T cells were incubated with about 0.5 μg-Fe of AAV-DJ chimeras. Scale bars, 10 μm.

FIG. 4B shows a confocal image of HEK293T cells fixed 24 hours after the addition of MNP-AAV chimeras (at a concentration of 0.5 μg of iron, 0.5 μg_{[Fe]}). Gray-Cytoplasm (CellTracker Orange), Green-NLS-GFP, Magenta-Cy7 attached to MNPs. Scale bar, 10 μm.

FIG. 4C shows an illustration of a hypothesized delivery and transit process of MNP-AAV chimeras.

FIG. 4D shows a schematic of an in vitro magnetic guidance test with MNP-AAV chimeras. A magnetic field (MF) was applied to the MNP-AAV chimeras by placing a cone-shaped neodymium magnet (12.7 mm diameter×12.7 mm tall, approximately 1.4 T, grade N50) directly under a cell culture dish. HEK293T cells were incubated with AAV-DJ chimeras (0.1 μg_{[Fe]}) for 1 minute followed by a medium exchange.

FIG. 4E shows low magnification confocal images of HEK293T cells incubated with MNP chimeras with AAV-DJ-CAG::NLS-GFP in the presence (“+MF”) or absence (“−MF”) of MFs. The cross marks show the center of the magnet. Scale bars, 3 mm.

FIG. 4F shows enlarged images of rectangular regions shown in FIG. 4E. The x-axis indicates the distance from the magnet center.

FIG. 4G is a graph showing the relationship between averaged intensities of NLS-GFP in the both “+MF” (top curve) and “−MF” (middle curve) conditions and the distance from the magnet center. The bottom curve shows a finite element model of a MF gradient at 2 mm above a cone-shaped magnet.

FIG. 5A shows representative IVIS images of mice that were injected with CAP-B10 chimera (left), MNP control (middle), and PBS (right). 4-week-old C57BL/6 (JAX) were fed with alfalfa-free diet (TestDiet AIN-93M) for 2 weeks, then used in the in vivo experiment. Retro-orbital route was selected for intravenous injection. 33.0 μg-Fe of CAP-B10 chimeras and MNP-Cy7 was injected for the chimera group and the MNP group, respectively. 100 μL of sterile PBS was injected to the PBS control.

FIG. 5B shows confocal images of the brains and livers of CAP-B10 chimera-injected mice (10-week-old) which were perfused at 2 weeks post injection (2×10¹⁰ vg/mouse). AAV.CAP-B10 that were conjugated with MNPs packaged pAAV-CAG::NLS-GFP. Blue-DAPI, Green-NLS-GFP. Scale bars, 1 mm (brain) and 100 μm (liver).

FIG. 5C shows IVIS timeline plots of average Cy7 radiant efficiency measured in the head from the same three groups as in FIG. 5A (N=4, Student's t-test).

FIG. 5D is a graph showing the average Cy7 radiant efficiencies in the brain dissected at 24 hours post injection (N=4, Student's t-test). Representative images are shown under the cohort labels. Data points represent individual tissues.

FIG. 5E is a graph showing the delivery efficiencies of CAP-B10 AAV chimeras and MNP-Cy7 controls quantified from the ex vivo fluorescence imaging data shown in FIG. 5D (N=4, Student's t-test).

FIG. 5F shows representative z-projected high magnification confocal images in the thalamus of CAP-B10 chimera injected mice at 24 hours and 48 hours post injection. Blue-DAPI, Green-Vasculature (Tomato lectin-DyLight488), Magenta-MNP-Cy7. Scale bar, 30 μm.

FIG. 5G is a graph showing the relationship between the ratio of signal intensities detected in the brain and liver and the average ratio χ̅. The amounts of MNPs injected were 33.0 μg-Fe (1.65 mg-Fe/kg) for χ̅=0.51 and 2.0 μg-Fe (0.1 mg-Fe/kg) for the other conditions. (N=4 for χ̅=0.51, 1.56, and 1.87. N=2 for χ̅=2.52).

FIG. 5H illustrates the signal level in perfused livers in different cohorts of mice (N=3, Student's t-test). The radiant efficiency was normalized by the amount of MNPs injected (1.4-4.7 μg-Fe/mouse). Error bars and shaded areas represent S.E.M. * P≤0.05, ** P≤0.01, *** P≤0.005.

FIG. 6A illustrates coating methods that lead to challenges in reproducibility and uniformity in coating.

FIG. 6B illustrates that silica shell coating by Secondary Surfactant Support Silica Coating (S⁴C) technology is highly reproducible and works with a variety of nanoparticles of different sizes and compositions. Scale bars are 50 nm.

FIG. 7A shows TEM images of 20 nm MNPs. The images display bare nanoparticles (left), silica-coated nanoparticles through RMM (middle, labeled ‘−OAc’) and nanoparticles produced through the S⁴C method (right, labeled ‘+OAc’). Scale bars are 100 nm.

FIG. 7B shows TEM images of 24 nm MNPs. The images display bare nanoparticles (left), silica-coated nanoparticles through RMM (middle, labeled ‘−OAc’), and nanoparticles produced through the S⁴C method (right, labeled ‘+OAc’). Scale bars are 100 nm.

FIG. 7C shows TEM images of 28 nm MNPs. The images display bare nanoparticles (left), silica-coated nanoparticles through RMM (middle, labeled ‘−OAc’), and nanoparticles produced through the S⁴C method (right, labeled ‘+OAc’). Scale bars are 100 nm.

FIG. 7D shows TEM images of MNDs. The images display bare nanoparticles (left), silica-coated nanoparticles through RMM (middle, labeled ‘−OAc’), and nanoparticles produced through the S⁴C method (right, labeled ‘+OAc’). Scale bars are 100 nm.

FIG. 7E shows TEM images of QDs. The images display bare nanoparticles (left), silica-coated nanoparticles through RMM (middle, labeled ‘−OAc’), and nanoparticles produced through the S⁴C method (right, labeled ‘+OAc’). Scale bars are 100 nm.

FIG. 7F is a graph showing the hydrodynamic diameters of silica-coated 24 nm MNPs measured by dynamic light scattering (DLS). The MNPs were coated using either the conventional method (‘−OAc’) or the S⁴C method (‘+OAc’).

FIG. 7G is a graph showing the comparison of proportion of multi-core particles between silica-coated QDs produced by the conventional method and S⁴C.

FIG. 7H is a graph showing the relationship between the shell thickness and the concentration of TEOS in the silica coating solution in S⁴C (N=3).

FIG. 7I shows TEM images corresponding to each data point shown in FIG. 7H. Scale bars are 50 nm.

FIG. 8A is a graph showing the relation between shell thickness and concentration of oleic acid (N=3).

FIG. 8B shows a representative TEM image of silica-coated MNPs at an oleic acid concentration of 68 mM. Many silica nanospheres (approximately 8 nm) were observed. Scale bars are 100 nm.

FIG. 8C is a graph showing the change in hydrodynamic diameter during the silica shell coating process with (‘+OAc’) and without oleic acid (‘−OAc’), as measured by DLS.

FIG. 9A is a graph showing magnetization curves of bare MNPs and silica-coated MNPs. MNP: bare MNPs. −OAc: silica-coated MNPs by the conventional method. +OAc, about 0.7 nm shell: silica-coated MNPs by S⁴C with shell thickness of about 0.7 nm. +OAc, 7.6 nm: silica-coated MNPs by S⁴C with shell thickness of about 7.6 nm.

FIG. 9B is a zoomed in portion of the graph marked with a dotted rectangle in FIG. 9A.

FIG. 9C is a graph showing specific loss power (SLP) values of silica-coated MNPs.

FIG. 9D is a graph of the fluorescence intensities of dye-labeled silica-coated MNPs. The values were normalized by the mass of core MNPs (top panel). An image of corresponding dye-labelled MNPs under UV light is provided beneath the bar plot (bottom panel).

FIG. 9E is a graph showing the emission spectra of bare QD and silica-coated QD. The excitation wavelength was 400 nm. The fluorescence intensities were normalized by the mass of QDs. The two lines are offset.

FIG. 9F shows fluorescence images of HEK cells cultured with BG-functionalized QDs and control cells. The cell nuclei were stained with a green dye (BioTracker green). QDs were excited at 600 nm and imaged in the window of 625-655 nm. Scale bar is 10 μm.

FIG. 9G shows a statistical analysis of the fluorescence intensity from the QDs normalized by cell area. Significant difference was observed. The model used was the Kruskal-Wallis one-way ANOVA. (*** P<0.0001).

FIG. 10A shows AAV-PHP.eB, AAV-PHP.V1, AAV-CAP-B10, AAV.MaCPNS1, and AAV.MaCPNS2 that were developed based on AAV9 by substituting and/or inserting short peptides. MaCPNS1 and MaCPNS2 do not have any extra lysine compared with AAV9. PHP.eB, PHP.V1, and CAP-B10 have 1, 1, and 2 more lysine residues per virus protein monomer compared with AAV9, respectively.

FIG. 10B shows the VP3 of AAV-LK03 is almost identical to AAV-3B.

FIG. 10C shows a visualization of accessible lysine residues on the capsid of AAV9, AAV-DJ, AAV-3B, and AAV-PHP.eB.

FIG. 11 shows transduction with dye labeled AAV-DJ. AAV-DJ-CAG::mNeonGreen was labeled with Cy5 through NHS chemistry at different Cy5/lysine ratio in the NHS reaction (T). The expression of mNeonGreen became weaker at τ≥5, implying AAVs may have lost their functionality as a gene vector by over-modification with Cy5 dye. The AAV to cell ratio was 30,000. The cells were incubated with AAVs for 24 hours in DMEM with 10% FBS. Scale bars, 200 μm.

FIG. 12A shows representative fluorescence images of brain and liver slices of mice that were injected with AAV.CAP-B10-CAG::NLS-GFP-Tz, which were prepared at different Tz to accessible lysine ratios. The expression of NLS-GFP in the brain decreased at Tz/Lys=4, while that in the liver slightly increased.

FIG. 12B shows representative fluorescence images of gut (jejunum) and liver slices of mice transduced by systemically injected AAV.MaCPNS2-CAG::NLS-GFP-Tz, which were prepared at different Tz to lysine ratios. The expression level in the gut slightly decreased at Tz/Lys=4 indicating over-functionalization of MaCPNS2 impaired AAV's trait to deliver transgenes. The dose was 5×10¹¹ vg/mouse for both serotypes. The expression time was 3 weeks.

FIG. 13 is a graph showing the relationship between hydrodynamic diameter of MNP-AAV conjugates and Tz/Lys ratio. Average hydrodynamic diameter (dₕ) was measured by DLS approximately 10 minutes after MNP@SiO₂-TCO and AAV-Tz (AAV-DJ) were mixed. [AAV]=1.2 nM. The MNP/AAV ratio in the reaction was 10. No quencher molecule was added.

FIG. 14 is a graph showing In situ DLS measurement of the chimerization reaction. With no quencher, dₕ of MNP-AAV conjugates increased sharply right after mixed. dₕ decreased after it had peaked, which may correspond to the precipitation of aggregates, leading to the decrease in the average dₕ. With quencher molecules, the size of MNP-AAV conjugates became stable throughout the measurement (approximately 270 minutes). dₕ increased with Tz/Lys, implying AAV-Tz synthesized at higher Tz/Lys is more reactive. [AAV]=1.2 nM. The MNP to AAV ratio=10.

FIG. 15A is a graph showing the parameter studies of the chimerization reaction. DLS measurements were performed about 10 minutes after AAV-Tz and MNP@SiO₂-TCO were mixed with quenchers, the hydrodynamic diameter dₕ decreased significantly.

FIG. 15B is a graph showing the parameter studies of the chimerization reaction. DLS measurements were performed about 10 minutes after AAV-Tz and MNP@SiO₂-TCO were mixed. The average dₕ increased with MNP/AAV ratio in the chimerization chemistry, suggesting that adding too many MNPs may cause larger aggregation.

FIG. 15C is a graph showing the parameter studies of the chimerization reaction. DLS measurements were performed about 10 minutes after AAV-Tz and MNP@SiO₂-TCO were mixed. dₕ was not sensitive to the equivalent amount of mPEG-Tz quencher to the TCO groups on MNPs over the ratio of unity, regardless of Tz/Lys.

FIG. 16 shows low magnification TEM images of QD chimeras. QDs were synthesized using 30% TCO/70% mPEG coating on QD@SiO₂, τ=0.5, ρ=3.3, and 4×mPEG-Tz quencher. AAV.CAP-B10 was used as the AAV for FIG. 16.

FIG. 17 shows TEM images of chimeras with various serotypes. Tz/Lys=1. [AAV]=6 nM. MNP to AAV ratio=2.

FIG. 18A is a graph showing the serotype dependence of the chimerization chemistry. The numbers of accessible lysine residues per capsid for different serotypes are 660 for AAV-DJ, 600 for AAV9, 720 for AAV.CAP-B10, and 600 for AAV.MaCPNS2. The average AAV/MNP ratios in chimeras synthesized with different serotypes. Serotypes having more lysine residues on the external surface of capsid (e.g., DJ and CAP-B10) resulted in higher average AAV/MNP ratios than those with less lysine residues (e.g., AAV9 and MaCPNS2), demonstrating higher reactivity of DJ and CAP-B10 at fixed Tz/Lys in NHS chemistry with intact AAVs.

FIG. 18B is a graph showing the serotype dependence of the chimerization chemistry. The numbers of accessible lysine residues per capsid for different serotypes are 660 for AAV-DJ, 600 for AAV9, 720 for AAV.CAP-B10, and 600 for AAV.MaCPNS2. The percentage of free MNPs in purified chimera solution for each serotype. Smaller amount of free MNPs were found in the chimeras synthesized with DJ and CAP-B10 than those made of AAV9 and MaCPNS2, showing higher reactivity of AAV-DJ and CAP-B10. Reaction conditions: Tz/Lys=0.5, MNP/AAV in the chimerization reaction=1, and 3× equivalents of mPEG-Tz and 0.2× equivalents Cy7-Tz were used as quenchers. In the calculation of χ̅, free MNPs and aggregates were excluded from the calculation.

FIG. 19 shows titers of supernatant from the purification step after chimera synthesis. During the purification steps after chimerization of AAV-Tz and MNP@SiO₂-TCO, the synthesized chimeras were washed with DPBS-F68 to remove unreacted AAV-Tz. After each centrifugation, the supernatant was removed completely, and 100 μL of fresh DPBS-F68 was added. The titer of the supernatant was measured by real-time polymerase chain reaction. After purification, the washed chimeras were re-suspended in 100 μL DPBS-F68, and its titer was also measured. The titer decreased with centrifuge cycles, meaning unreacted AAV-Tz's were removed by the purification. The removal of unreacted free AAV-Tz was also confirmed by TEM images. The titer of chimera was ˜100 times greater than the 4^{th} supernatant, suggesting that AAVs conjugated to MNPs were spun down with MNPs and not washed away with free AAV-Tz's. This result is also quantitative evidence of the conjugation of AAVs to MNPs.

FIG. 20 shows transmission electron micrographs of purified chimera mixtures. AAV.CAP-B10 chimeras, unreacted free MNPs, and aggregates (unlabeled circles) are indicated on the graph.

FIG. 21 is a graph showing the influence of Tz/Lys ratio on chimerization. The average χ (χ̅, black) increased with t, while the percentage of aggregates (light blue) increased together and the percentage of free MNPs (green) decreased. There is a trade-off between the free MNP % and aggregate %.

FIG. 22 shows sample low magnification transmission electron micrographs of AAV-DJ chimeras synthesized at different AAV/MNP ratio ρ in the conjugation chemistry. The corresponding population break down of each ratio of AAV/MNP in chimeras (χ) is in FIG. 2D.

FIG. 23A is a graph showing the gating and results for the delivery specificity test of AAV serotypes to the HEK293 cell line by flow cytometry. Based on this result, serotypes that demonstrated targeting specificity to these cell lines (e.g., LK03×HeLa, PHP.V1×C1C12) were employed in the targeting specificity test of chimeras (FIG. 3C). Cells were incubated with AAVs for 24 hours, then the fluorescence from mRuby2 was measured without being fixed.

FIG. 23B is a graph showing the gating and results for the delivery specificity test of AAV serotypes to the HeLa cell line by flow cytometry. Based on this result, serotypes that demonstrated targeting specificity to these cell lines (e.g., LK03×HeLa, PHP.V1×C1C12) were employed in the targeting specificity test of chimeras (FIG. 3C). Cells were incubated with AAVs for 24 hours, then the fluorescence from mRuby2 was measured without being fixed.

FIG. 23C is a graph showing the gating and results for the delivery specificity test of AAV serotypes to the C2C12 cell line by flow cytometry. Based on this result, serotypes that demonstrated targeting specificity to these cell lines (e.g., LK03×HeLa, PHP.V1×C1C12) were employed in the targeting specificity test of chimeras (FIG. 3C). Cells were incubated with AAVs for 24 hours, then the fluorescence from mRuby2 was measured without being fixed.

FIG. 24A is a graph showing the averaged mean intensity of MNP-Cy7 signal for all combinations of AAV serotypes and the HEK293 cell line. Black dots are the averaged MNP-Cy7 signal intensity from each sample. The grey dots are the means of black dots. The error bars are S.E.M.

FIG. 24B is a graph showing the averaged mean intensity of MNP-Cy7 signal for all combinations of AAV serotypes and the HeLa cell line. Black dots are the averaged MNP-Cy7 signal intensity from each sample. The grey dots are the means of black dots. The error bars are S.E.M.

FIG. 24C is a graph showing the averaged mean intensity of MNP-Cy7 signal for all combinations of AAV serotypes and the C2C12 cell line. Black dots are the averaged MNP-Cy7 signal intensity from each sample. The grey dots are the means of black dots. The error bars are S.E.M.

FIG. 25 shows heat maps visualizing mRuby2+% and MNP-Cy7+%. Targeting specificity test with different cell lines and AAV serotypes is visualized as heat maps. The top row of each cell line shows mRuby2+% after 24 hour long transduction with intact AAVs. The second row of each cell line shows Cy7+% after 4 hour incubation.

FIG. 26 shows 3D reconstructions of confocal images in FIG. 4A. Blue-DAPI (nucleus), Green-membrane (CellMask Deep Red), Magenta-MNP-Cy7. Z-scan was performed with a step of 0.3 μm.

FIG. 27A shows line profiles of live-cell confocal images in FIG. 4A at 10 minutes. Line profile analysis was performed on Fiji/ImageJ. Blue-DAPI. Green-CellMask membrane dye. Magenta-MNP-Cy7.

FIG. 27B shows line profiles of live-cell confocal images in FIG. 4A at 2 hours. Line profile analysis was performed on Fiji/ImageJ. Blue-DAPI. Green-CellMask membrane dye. Magenta-MNP-Cy7.

FIG. 28 shows ex vivo IVIS images of the brain and liver dissected from the mice used in the in vivo fluorescence imaging (FIGS. 5B-5E). About 41.2 μg_{[Fe]}/mouse (2.06 mg_{[Fe]}/kg) of CAP-B10 chimera and MNP-Cy7 was injected to the relevant groups. The mice were perfused at 24 hours post injection.

FIG. 29 is a graph showing the standard curve of ex vivo fluorescence imaging. A dilution series of MNP-Cy7 mixed with 1% agarose gel (1.5 mL) was imaged using the same ex vivo fluorescence imaging machine and settings as tissue samples (FIG. 5D).

FIG. 30A shows low magnification TEM images of CAP-B10 chimeras. Approximately 50% of MNPs in the solution was AAV-free MNPs.

FIG. 30B shows low magnification TEM images of CAP-B10 chimeras. Approximately 50% of MNPs in the solution was AAV-free MNPs.

FIG. 30C shows quantification of the same CAP-B10 chimera in FIGS. 30A and 30B. Approximately 50% of MNPs in the solution was AAV-free MNPs.

FIG. 31 shows tiled 10× magnification confocal images of the same brain slice as FIG. 5F. Tomato-lectin-DyLight 488 was injected 10 minutes before perfusion. The slice was stained with DAPI for 20 minutes. Scale bars, 1 mm.

FIG. 32A shows tiled 20× and 63× magnification confocal images of the same mouse brain slices as FIG. 5F (24 hours). In the Hippocampus MNP-Cy7 colocalizes with large vasculatures at this magnification. Scale bars, 500 μm.

FIG. 32B shows tiled 20× and 63× magnification confocal images of the same mouse brain slices as FIG. 5F (24 hours). In the Thalamus. MNP-Cy7 exists in capillaries. Scale bars, 20 μm.

FIG. 33 shows 63× magnification confocal images of the same slice as FIG. 5F (48 hours) and PBS control. Left: Somatosensory areas layer 5, Right: Ventral posteromedial nucleus of the thalamus. Approximately 5 μg Fe of MNPs was injected (0.25 mg Fe/kg-animal). Vasculatures were stained with 100 μL of Tomato lectin-DyeLight 488, which was injected intravenously 10 minutes before perfusion. Scale bars, 20 μm.

FIG. 34A shows low magnification TEM images of CAP-B10 chimeras with four different χ̅ values.

FIG. 34B is a graph showing the quantification of the same samples in FIG. 34A.

FIG. 34C is a graph showing the percentage of AAV-free MNPs and aggregates that visualized in FIG. 34A.

FIG. 35 shows ex vivo fluorescence images of the liver and the brain used in the quantification for FIG. 5G. The fluorescence from Cy7 attached to MNPs was detected (Excitation filter=745 nm, emission filter=800 nm). The mice were perfused 24 hours post injection. The amounts of MNPs injected were approximately 33.0 μg Fe (1.65 mg Fe/kg) for χ̅=0 and 0.51, and 2.0 μg Fe (0.1 mg Fe/kg) for the other conditions.

FIG. 36 shows TEM image (left panel) and analyses (right panel) of AAV9 chimera. Tz/Lys=1.0 (AAV9. [AAV]=6.0 nM. The AAV/MNP ratio in chimerizaiton was 10. Scale bar, 100 μm.

FIG. 37 is a graph showing the quantification of fluorescence signal in the brain of mice injected with AAV9 chimera or MNP control. The total fluorescence in the brain was quantified and statistically tested (Student's t-test, p=0.618, N=3). The radiant efficiency from Cy7 was normalized by the amount of MNPs injected (1.4-4.7 μg_{[Fe]}/mouse).

### DETAILED DESCRIPTION

The systemic delivery of nanoparticles (NPs) has garnered significant attention from researchers due to its clinical promise. Various strategies for delivering NPs have been developed thus far, including the utilization of the enhanced permeability and retention (EPR) effect to target tumors or the modification of NPs with antibodies to target specific proteins. However, delivering NPs to the brain through the blood-brain barrier (BBB) remains a challenge. Additionally, the importance of the communication between the central nervous system (CNS) and the peripheral nervous system (PNS) has been increasingly recognized, leading to a high demand for delivery systems that can flexibly change their target between the CNS and PNS. Translation of delivery strategies between species offers another challenge, especially in the context of bridging the divide between fundamental research in rodents and clinical research in larger models such as non-human primates (NHPs). Therefore, there is a pressing need for systemic delivery of nanoparticles that is both translational and capable of flexibly selecting targets.

For decades the field of gene therapy has applied engineered viruses to deliver therapeutic transgenes to particular organs or cells of interest. Adeno-associated viruses (AAVs) are the most ubiquitous gene-therapy tools, particularly in neuroscience, owing to their advantageous characteristics: AAVs are non-pathogenic, easily reproducible, and have numerous accessible serotypes, each with distinct tropism. State-of-the-art serotypes, such as AAV.CAP-B10 and AAV.MaCPNS2, can deliver genes to the brain or PNS in mice following intravenous (IV) injection, while simultaneously suppressing gene expression in the liver. These serotypes have also shown promise for gene delivery in NHPs.

### Gene Targeting Chimeras

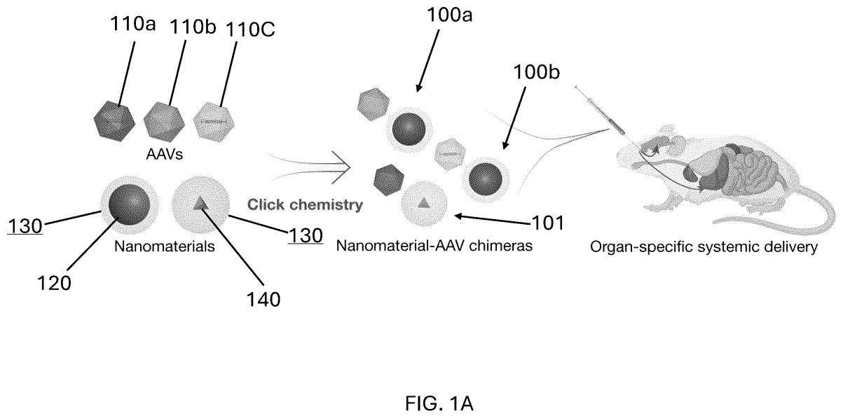

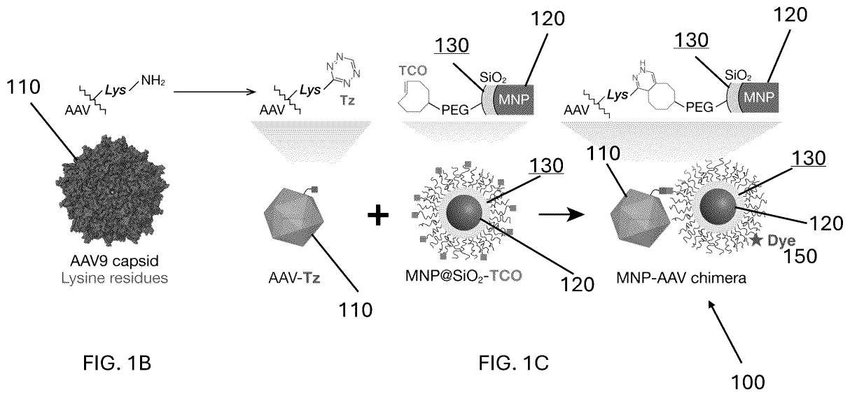

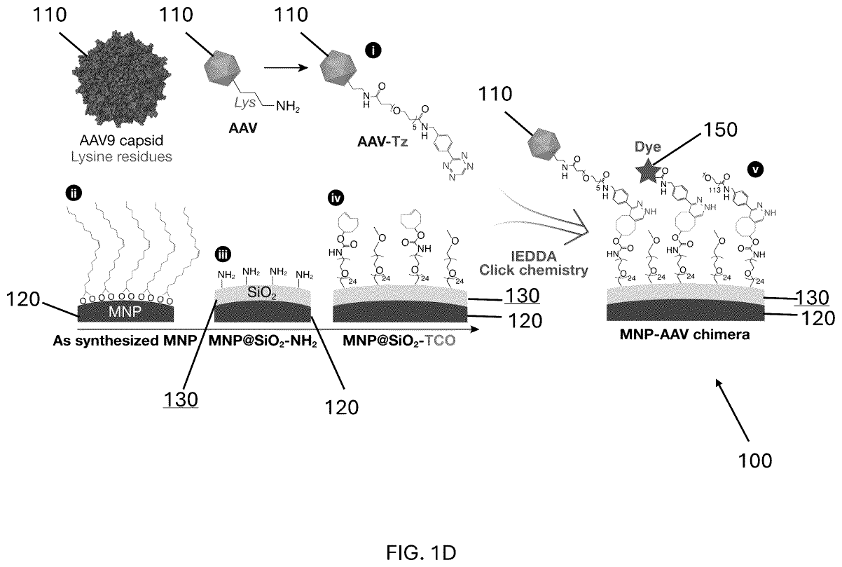

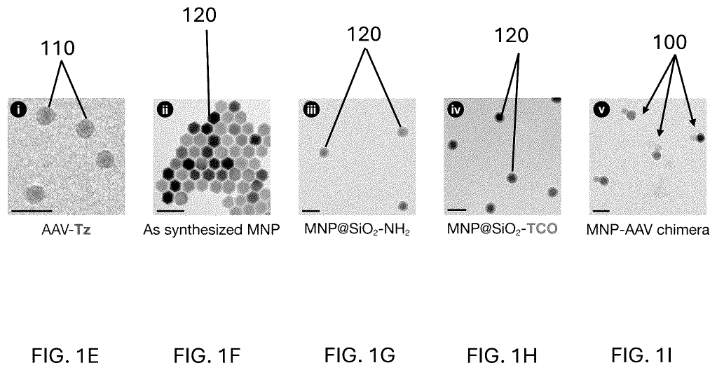

FIGS. 1A-9G show how AAVs can be employed to guide nanoparticles (e.g., magnetic nanoparticles (MNPs), magnetic nanodics (MNDs), or quantum dots (QD)) to particular organs through IV injection. In one embodiment, the nanoparticle may be an MNP. The MNPs may range in size from about 10 nm to about 40 nm in diameter, preferably about 20 nm to about 25 nm in diameter. The MNPs may be either spherical or faceted in shape. The MNPs may be made of magnetite (Fe₃O₄), Wüstite (FeO), hematite (Fe₂O₃), or another suitable material. The MNP may be attached to one or more AAVs to form a gene targeting chimera (also called an MNP-AAV chimera or chimera, where a chimera is an engineered construct in which two or more components are linked to form a novel biological agent). For example, the MNP may be attached to one, two, three, four, five, six, seven, eight, nine, or ten AAVs. Preferably, the MNP is attached to more than one AAV, for example, two, three, four, five, six, seven, eight, nine, or ten AAVs.

The MNP-AAV chimeras may be gene-targeting chimeras. The MNP-AAV chimeras may allow for modification of a target location in a mammal by swapping AAV serotypes. For example, one MNP may be conjugated with an AAV serotype that targets the brain, another MNP may be conjugated with an AAV serotype that targets the PNS, and yet another MNP may be conjugated with an AAV serotype that targets the liver. Thus, the targeted cell location may be chosen by selecting the appropriate AAV. The MNP-AAV chimeras may be guided to a target location in a mammal using a magnetic field. The use of MNP-AAV chimeras may also be less invasive than the delivery of AAVs directly to the target area. Furthermore, by selecting an appropriate AAV serotype, particular organ(s) in different animals can be targeted. This concept, termed viral guidance of MNPs, has the potential to serve as a versatile targeting strategy. The AAVs may allow targeting of certain organs (e.g., the brain) and the MNPs may allow for the targeting of a specific part of an organ (e.g., the hippocampus in the brain) through the use of a magnetic field. Thus, the MNP-AAV chimeras disclosed herein may allow for both the targeting of a specific organ(s) and a specific part(s) of said organ.

Instead of, or in addition to, a MNP, an AAV may be attached to another nanoparticle. For example, one or more AAVs may be attached to a QD (also called an QD-AAV chimera or QD chimera). The QD may be conjugated with one, two, three, four, five, six, seven, eight, nine, or ten AAVs. Preferably, the QD is attached to more than one AAV, for example, two, three, four, five, six, seven, eight, nine, or ten AAVs. The QD chimeras may be gene-targeting chimeras. As described above, the conjugation of a QD and an AAV(s) may allow for modification of a target location in a mammal by swapping AAV serotypes. QD chimeras may also be guided to a target location in a mammal using a magnetic field.

The nanoparticle may also be a magnetic nanodic (MND). One or more AAVs may be attached to an MND to form an MND-AAV chimera. The MND may be conjugated with one, two, three, four, five, six, seven, eight, nine, or ten AAVs. Preferably, the MND is attached to more than one AAV, for example, two, three, four, five, six, seven, eight, nine, or ten AAVs. The MND-AAV chimera may be a gene-targeting chimera. As described above, the conjugation of a MND and an AAV(s) may allow for modification of a target location in a mammal by swapping AAV serotypes. The MND-AAV chimera may also be guided to a target location in a mammal using a magnetic field.

In yet another embodiment, the AAV may be attached to a drug and/or an imaging agent (e.g., a dye), through the same synthetic scheme described below. An MNP-AAV chimera, a QD chimera, and/or an MND-AAV chimera may also be modified with a drug and/or an imaging agent as further described below.

MNP-AAV chimeras, QD chimeras, and/or MND-AAV chimeras may be covalently attached or conjugated through a linking chemistry, for example, click chemistry or another suitable linking chemistry. For the click chemistry, the Inverse Electron-Demand Dials-Alder (IEDDA) reaction may be used because it has a large rate constant, which may allow for the formation of covalent bonds between the MNPs, MNDs, or QDs and AAVs. The IEDDA reaction is a catalyst-free click reaction that may have pronounced chemoselectivity and fast reaction rates. The structure of these gene targeting chimeras may be controlled by adjusting the reaction conditions, such as the number of functional groups for IEDDA reaction on both the MNPs or QDs and AAVs. Both the MNP, MND, QD, and AAVs may be modified with complementary functional groups for an IEDDA reaction, for example, tetrazine and trans-cyclooctene. The MNPs, MNDs, QDs, and AAVs may be modified with either one of the complementary functional groups for an IEDDA reaction.

The MNP-to-AAV ratio, MND-to-AAV ratio, and/or the MNP-to-QD ratio may also be controlled by adjusting the reactivity (e.g., kinetics) of the click reaction to create a variety of MNP-AAV chimeras, MND-AAV chimeras, and/or QD chimeras with different ratios of MNPs, MNDs, or QDs to AAVs, from 1:1 to 1:8. For example, the MNP:AAV ratio may be 1:1, 1:2, 1:3, 1:4, 1:5, 1:6, 1:7, or 1:8. The QD:AAV ratio may be 1:1, 1:2, 1:3, 1:4, 1:5, 1:6, 1:7, or 1:8. The MND:AAV ratio may be 1:1, 1:2, 1:3, 1:4, 1:5, 1:6, 1:7, or 1:8. The MNP-to-AAV ratio, MND-to-AAV ratio, and/or the MNP-to-QD ratio may be controlled using a quenching agent (e.g., tetrazine-methoxy polyethylene glycol or trans-cyclooctene methoxy polyethylene glycol) that titrates the rate of the reaction. Prior to conjugating the MNPs, MNDs, and/or QDs and the AAVs, the MNPs, MNDs, and/or QDs may be standardized using a silica shell coating as described below. The silica shell coating may result in MNPs, MNDs, and/or QDs with essentially the same surface. The silica shell coating may be about 0.7 nm to about 20 nm, about 2 nm to about 8 nm thick, preferably about 4 nm thick.

The serotype of the AAVs may guide the MNP-AAV chimeras, MND-AAV chimeras, and/or QD chimeras to a particular location after insertion into a mammal. In vitro tests were conducted to investigate the efficiency of AAVs in guiding MNPs, MNDs, and/or QDs using MNP-AAV chimeras, MND-AAV chimeras, and/or QD chimeras formed from several different AAV serotypes. MNP-AAV chimeras, MND-AAV chimeras, and/or QD chimeras may retain the tropism of the original AAV serotype used, indicating that the delivery target may be serotype-dependent and may be easily modified by changing the AAV serotype used for conjugation. MNP-AAV chimeras, MND-AAV chimeras, and/or QD chimeras may be contained in a solution. The solution may include a plurality of gene targeting chimeras. The solution of MNP-AAV chimeras, MND-AAV chimeras, and/or QD chimeras may be injected intravenously into a mammal. For example, for injection into a mouse, a solution of MNP-AAV chimeras may include about 1-10*10¹⁰ AAVs and about 10 μg to 100 μg of MNPs. Similarly, for injection into a mouse, a solution of QD chimeras may include about 1-10*10¹⁰ AAVs and about 10 μg to 100 μg of QDs. Similarly, for injection into a mouse, a solution of MND-AAV chimeras may include about 1-10*10¹⁰-11 AAVs and about 10 μg to 1000 μg of MNDs.

The MNP-AAV chimeras and/or MND-AAV chimeras may also be able to be controlled using a magnetic field. For example, a magnetic field may be used to localize and target specific regions in the body. The MNPs and MNDs in MNP-AAV and/or MND-AAV chimeras can also be used for magnetic guidance of AAVs. Since AAVs were conjugated to MNPs or MNDs, the MNP-AAV and/or MND-AAV chimeras may be manipulated using an external magnetic field to exert a magnetic force on the MNPs and/or MNDs. For example, as is described in more detail below, HEK293 cells were incubated with MNP-AAV chimeras in the presence of a magnetic field. Following a 24-hour incubation period, the HEK cells expressed high levels of fluorescent protein, which was encoded by the AAVs in the MNP-AAV chimeras, near the center of the magnetic field. This result suggests that magnetic guidance of AAVs is feasible, and that AAVs in a chimera form (e.g., MNP-AAV chimeras) may be capable of transducing cells. Similarly, an MND-AAV chimera may also be utilized to magnetically guide AAVs. The specificity of gene delivery targeting with AAV was previously determined by a combination of two factors: serotype (capsid) and regulatory elements (promoter, enhancer, Cre-lox system, etc.). Now, the MNP-AAV and MND-AAV chimeras disclosed herein may enable spatial restriction of transduction, which adds another layer of control to gene delivery.

AAVs may be leveraged as guides for the systemic delivery of nanoscale payloads by exploiting their natural tropism (FIG. 1A). The MNP-AAV chimeras, MND-AAV chimeras, and/or QD chimeras disclosed herein may be used to test this theory. In vitro, these MNP-AAV chimeras, MND-AAV chimeras, and/or QD chimeras may selectively escort nanoparticles into the specific cells according to the tropism of AAVs. Additionally, these MNP-AAV and/or MND-AAV chimeras may maintain the sensitivity of MNPs and MNDs to magnetic field gradients to permit spatial restriction of gene expression. For example, an MNP-AAV chimera (e.g., a AAV.CAP-B10 chimera) with precisely controlled MNP-AAV stoichiometry can be used for efficient and specific AAV delivery in the mouse brain. These MNP-AAV chimeras achieved a delivery efficiency of approximately 4% ID (˜10% ID/g brain) in wild-type mice, which is approximately 3 times higher than previous reports on molecular cargo (<4% ID/g brain) and over 10 times higher than previous reports for nanoparticles (<1% ID/g brain). Furthermore, these MNP-AAV chimeras, MND-AAV chimeras, and/or QD chimeras may effectively avoid accumulation in the liver while retaining their gene delivery function. Given the vast and rapidly growing array of viral vectors, the findings disclosed herein may unlock a promising strategy to systemically deliver large therapeutic cargos to specific tissues and cells.

FIG. 1A shows various gene targeting chimeras that may be injected into a mammal for organ-specific systemic delivery. The nanoparticle-AAV chimera may be an MNP-AAV chimera 100. The MNP-AAV chimera 100 may include one or more AAVs 110. Each AAV 110 may be a unique serotype. For example, AAV 110a may be one serotype, AAV 110b may be a second serotype, and AAC 110c may be a third serotype. The MNP-AAV chimera 100 may also include an MNP 120. The MNP 120 made of magnetite, Fe₃O₄, or another suitable material. The MNPs 120 may be synthesized as described below. The MNP 120 may range in size from about 10 nm to about 40 nm in diameter, preferably about 20 nm to about 25 nm in diameter. The MNP 120 may be either spherical or faceted in shape. The MNP 120 may also include a silica shell coating 130. The silica shell coating 130 may be approximately 2 nm to 8 nm thick, preferably about 4 nm thick, and may be formed on the MNP 120 as described below. The silica shell coated MNP 120 may be conjugated to the AAV 110 via a linking chemistry (e.g., click chemistry) to form the MNP-AAV chimera 100. Each MNP-AAV chimera 100 may target a specific organ and/or part of a specific organ based on the AAV 110 serotype. For example, MNP-AAV chimera 100a may target a first organ and/or part of a first organ and MNP-AAV chimera 100b may target a second organ and/or part of a second organ.

Alternatively, the nanoparticle-AAV chimera may be a QD chimera 101. The QD chimera 101 may include a QD 140. The QD 140 may be made of cadmium selenide and/or zinc sulfide, or another suitable material. The QD 140 may range in size from about 5 nm to about 20 nm in diameter, preferably about 8 nm to about 15 nm in diameter. The QD 140 may also include a silica shell coating 130 as described above. The silica shell coating 130 may be approximately 2 nm to 20 nm thick, preferably about 4 nm thick to about 15 nm thick, and may be formed on the QD 140 as described below. The silica shell coated QD 140 may be conjugated to the AAV 110 via a linking chemistry (e.g., click chemistry) to form the QD chimera 101. Each QD chimera 101 may target a specific organ and/or part of a specific organ based on the AAV 110 serotype.

### Design of Chimerization Chemistry

First, a scheme for conjugating nanoparticles with AAVs was developed. Magnetite (Fe₃O₄) MNPs 120 were initially selected for their ubiquitous use in biomedical and imaging applications. The diameter of the MNPs 120 may be approximately 10 nm to 40 nm, for example, about 24.0±1.8 nm. Preferably the diameter of the MNP 120 may be similar to the diameter of an AAV 110 capsid. To ensure efficient conjugation at low molar concentrations characteristic of large-particle solutions, the inverse electron-demand Diels-Alder (IEDDA) reaction for chimerization chemistry (rate constant, k=1-10⁶ M⁻¹s⁻¹) was used. AAVs 110 were modified with tetrazine (Tz) and MNPs 120 were functionalized with trans-cyclooctene (TCO) groups that formed a covalent bond during chimerization (FIGS. 1C and 1D). Alternatively, the MNPs 120 may be modified with Tz and the AAVs 110 may be modified with TCO groups. Other suitable functional groups may also be used to modify the AAVs 110 and MNPs 120.

Adeno-associated viruses (AAVs) and magnetic nanoparticles (MNPs) are both approximately 24 nm in diameter. These “particles” are larger than molecules (approximately 100 nm) and proteins (approximately 10¹ nm), and the reaction rate between them is typically slow. For instance, a common titer of AAV packaged by a standard protocol is 10¹³ viral genome (vg) per mL, which corresponds to 16.6 nM. Considering the potential loss of virus in the chemical modification step prior to conjugation and the volumes of other reactants, the reaction concentration may be on the order of 1 nM or less. The conjugation reaction is MNP+AAV→MNP−AAV, and the reaction rate is given by Equation 1:

[MNP−AAV]=k[MNP][AAV] (Equation 1),

when it is reaction-limited, where [MNP] means the concentration of MNP and k is a rate constant. Therefore, when the concentration of MNPs and AAVs is very small, a large rate constant may be needed to obtain the product, MNP-AAV.

This reaction is not considered to be diffusion-limited. Diffusion-limited reactions may happen when a reaction consumes a large number of reactants and causes an inhomogeneous distribution of reactants, such as crystallization of solute at a surface. In this case, an MNP may only consume about 1-8 AAVs, and the concentration of AAVs near an MNP may not be significantly affected by that. Therefore, it can be assumes that the distribution of particles is not biased enough to make the diffusion rate constant smaller than the reaction rate constant.

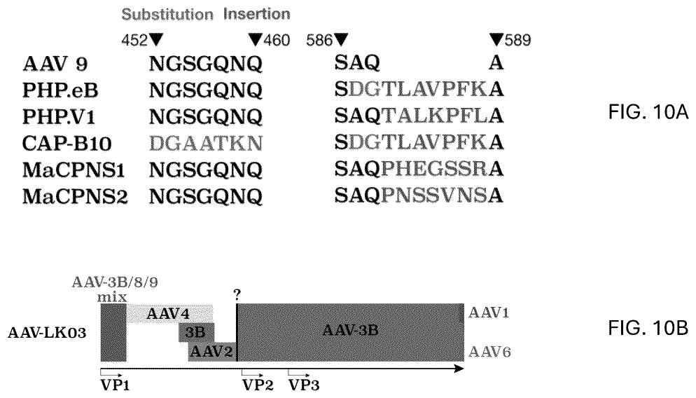

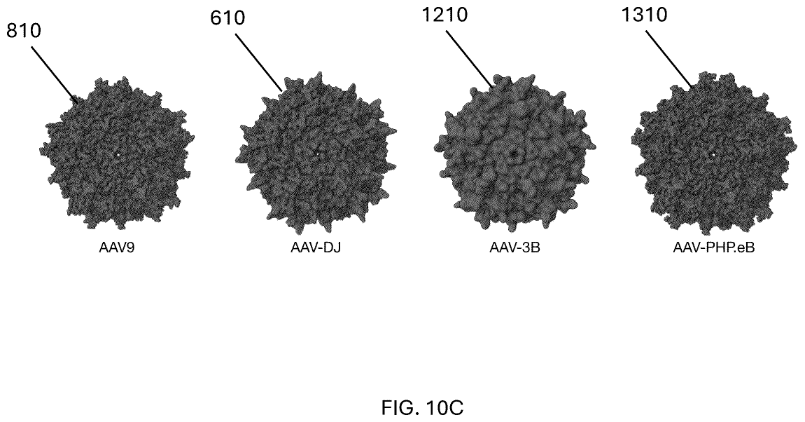

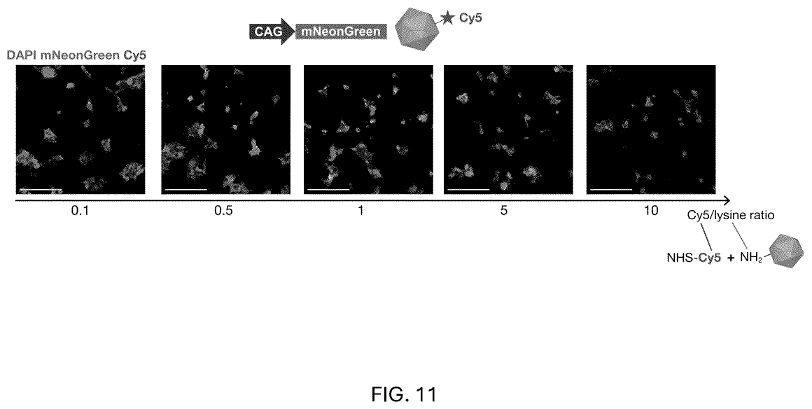

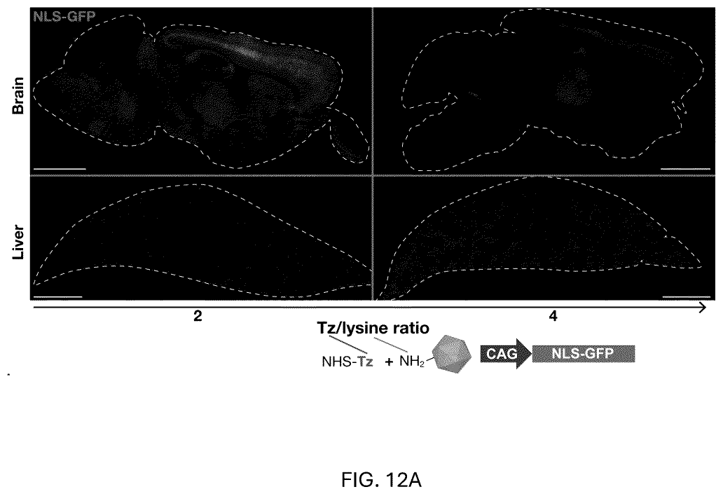

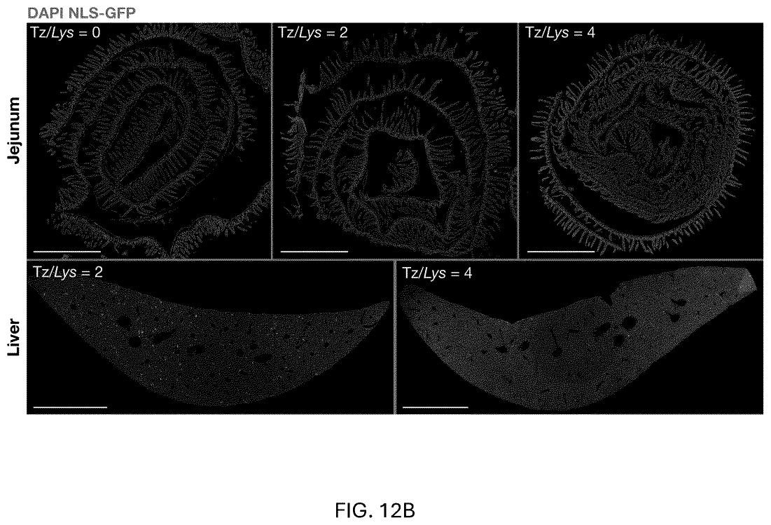

Chemical modification of AAVs 110 with small molecules, polymers, and multichelators has been reported previously. However, over-modification of AAV 110 capsids may impair their gene carrier function. Here, Tz-functionalized AAVs 110 (AAV-Tz) were prepared through carbodiimide chemistry with primary amine groups in lysine residues (Lys) on the AAV 110 capsids since many AAV 110 serotypes possess a similar number of accessible Lys, making this strategy generalizable across a wide range of AAV 110 serotypes (FIGS. 1B, 10A-10C, and Table 1). Although some exposed Lys may play a role in AAV 110 tropism, it was previously reported that the residues contributing to engineered AAVs' 110 tropism may be less likely to participate in the carbodiimide coupling. The functionality of chemically modified AAVs 110 as viral vectors in vitro (FIG. 11) as well as their tropism in vivo was confirmed (FIGS. 12A-12B). AAVs 110 modified with Tz lose their targeting tropism at the 2:1 ratio of Tz-NHS to Lys in the reaction solution. Thus, in the following sections, a 1:1 ratio of Tz-NHS to Lys was used for AAV 110 modification unless otherwise specified.

TABLE 1
Number of accessible lysine residues per capsid
in different AAV serotypes

| AAV serotype | # accessible Lys/capsid | Reference |
|---|---|---|
| AAV9 810 | 600 | PDB ID: 3UX1 |
| AAV-DJ 610 | 660 | PDB ID: 7KFR |
| AAV-LK03* | 480 | PDB ID: 3KIC |
| AAV-PHP.V1** 1310 | 660 | |
| AAV.CAP-B10** | 720 | |
| AAV.MaCPNS1, 2** | 600 | |

*The number of accessible lysine residues per capsid was estimated from that of AAV-3B 1210. The capsid protein VP3 of AAV-LK03 is almost identical to that of AAV-3B 1210.
**The number of accessible lysine residues per capsid was estimated based on that of AAV9.

### Control Over Chimera Stoichiometry

FIGS. 1C and 1D illustrate the functionalization of AAVs 110 and MNPs 120 to enable the subsequent chimerization using an IEDDA reaction. MNPs 120 with a diameter of about 24 nm were synthesized using the thermal decomposition method, resulting in a hydrophobic surface capped with oleic acid (FIG. 1D, panels i-ii and FIGS. 1E and 1F). MNPs 120 were then coated with 2.9±0.7 nm silica shells 130 (MNP@SiO₂) via the silica coating with hydrophobic ligand in reverse microemulsion method. The MNP@SiO₂ particles were then functionalized with amines (MNP@SiO₂—NH₂. FIG. 1D, panel iii and FIG. 1G) and subsequently with TCO (MNP@SiO₂-TCO, FIG. 1D, panel iv and FIG. 1H). The final diameter of MNP@SiO₂-TCO determined by transmission electron microscopy (TEM) was approximately 29.8±2.2 nm.

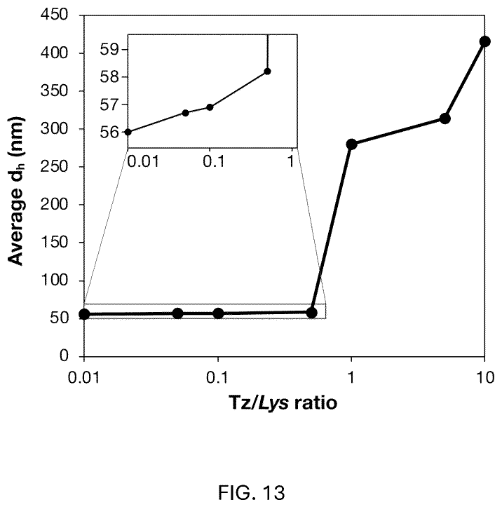

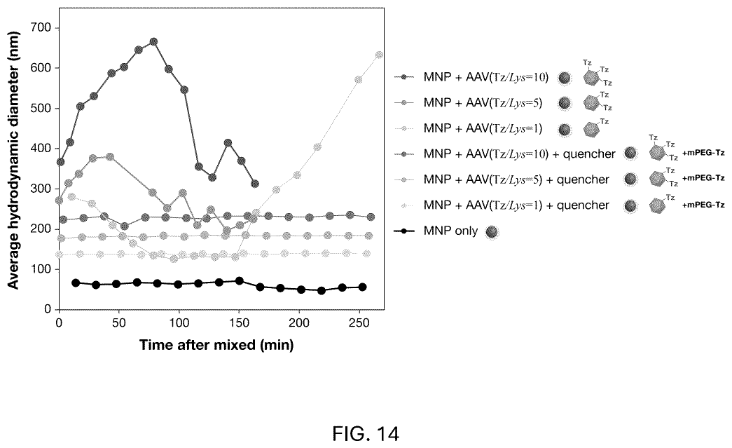

Finally, MNP@SiO₂-TCO and AAV-Tz were mixed and covalently conjugated through an IEDDA reaction (FIG. 1D, panel v). Monitoring the reaction with in situ dynamic light scattering showed that upon mixing the two particles reacted rapidly and aggregated within approximately 10 minutes (see FIGS. 1A-1J and 13). Therefore, to prevent aggregation, a quencher molecule (mPEG-Tz (5 kDa)) was employed to inactivate the TCO groups on MNP@SiO₂-TCO (see FIGS. 13 and 14). To facilitate MNP 120 visualization with optical microscopy, an infrared dye 150 (e.g., Cy7 fused to Tz (Cy7-Tz)) was also employed during a subset of experiments, where it may serve as an additional quencher (FIGS. 1C and 1D).

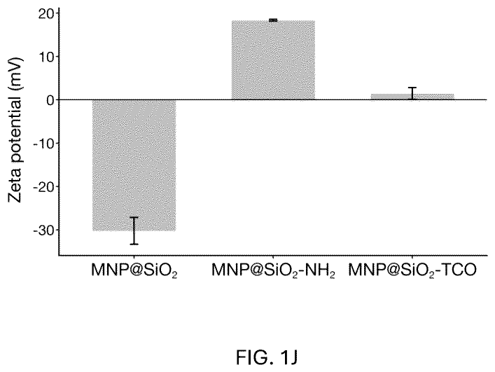

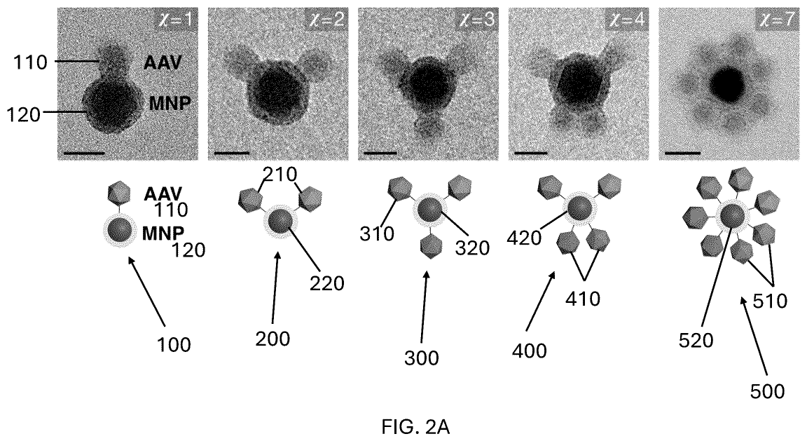

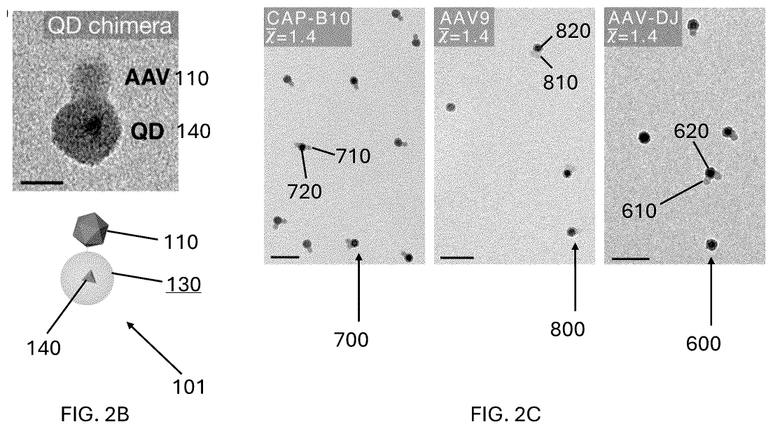

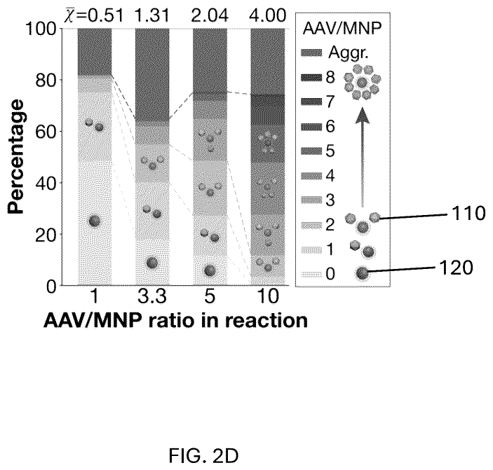

FIG. 2A illustrates MNP-AAV chimeras 100 with different AAV-to-MNP ratios (denoted χ). The conjugation reaction rate for the MNP-AAV chimeras 100 may be governed by the stoichiometry of AAV-Tz, quenchers, and MNP@SiO₂-TCO, enabling precise control over the AAV-to-MNP ratio (χ) in the resulting MNP-AAV chimeras 100. As shown in FIG. 2A, MNP-AAV chimera 200 may have an AAV-to-MNP ratio of 2, MNP-AAV chimera 300 may have an AAV-to-MNP ratio of 3, MNP-AAV chimera 400 may have an AAV-to-MNP ratio of 4, and MNP-AAV chimera 500 may have an AAV-to-MNP ratio of 7. Each MNP-AAV chimera 200, 300, 400, 500 may include an MNP 220, 320, 420, 520, 620. The MNPs 220, 320, 420, 520, and/or 620 may include a silica shell coating 130 as described above. The MNPs 220, 320, 420, 520, and/or 620 may be attached to one or more AAVs 210, 310, 410, 510, and/or 610, respectively. For example, as shown in FIG. 2A, MNP-AAV chimera 200 may include two AAVs 210, MNP-AAV chimera 300 may include three AAVs 310, MNP-AAV chimera 400 may include four AAVs 410, and MNP-AAV chimera 500 may include five AAVs 510. The AAVs 210, 310, 410, 510, and/or 610 may be attached to the MNPs 220, 320, 420, 520, and/or 620 as described above.

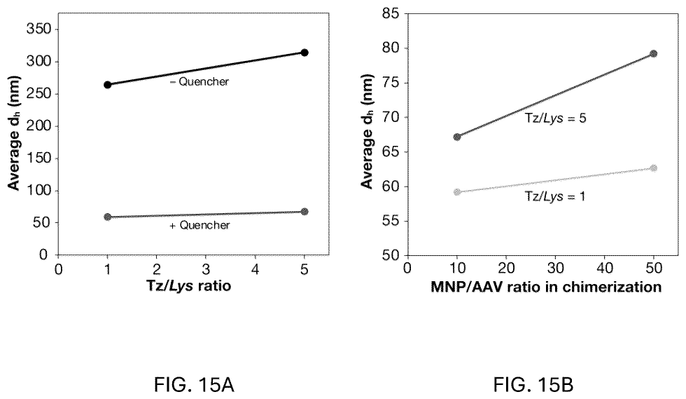

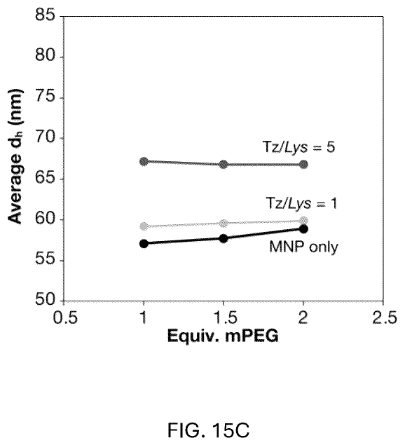

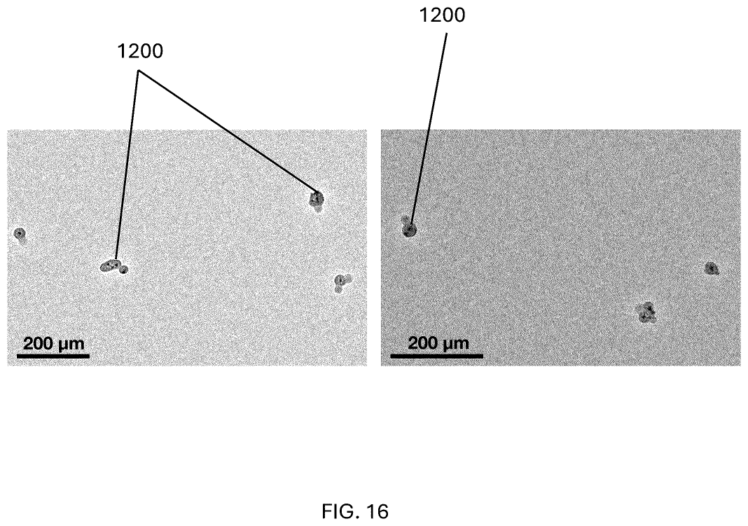

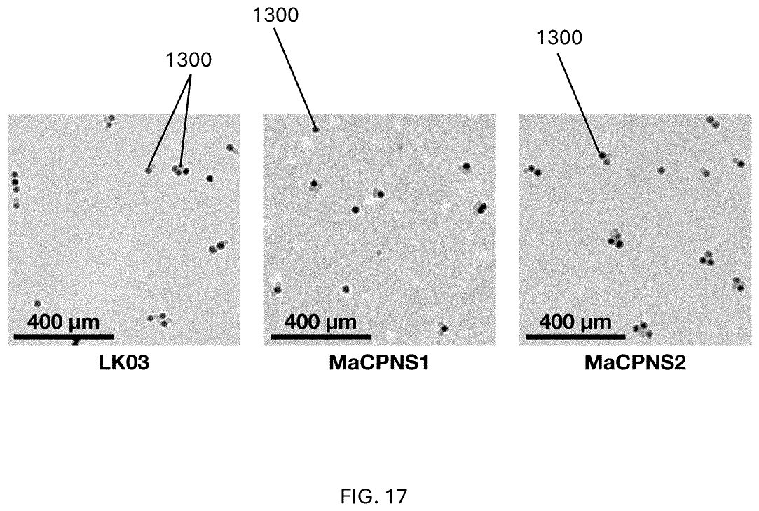

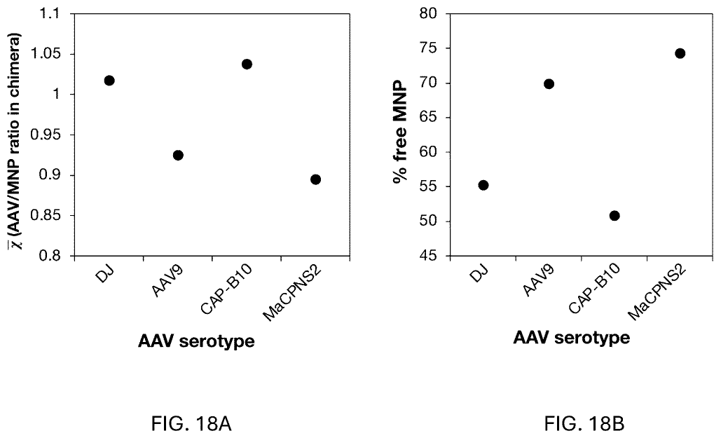

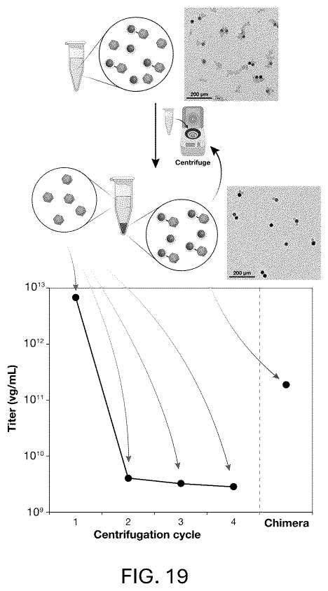

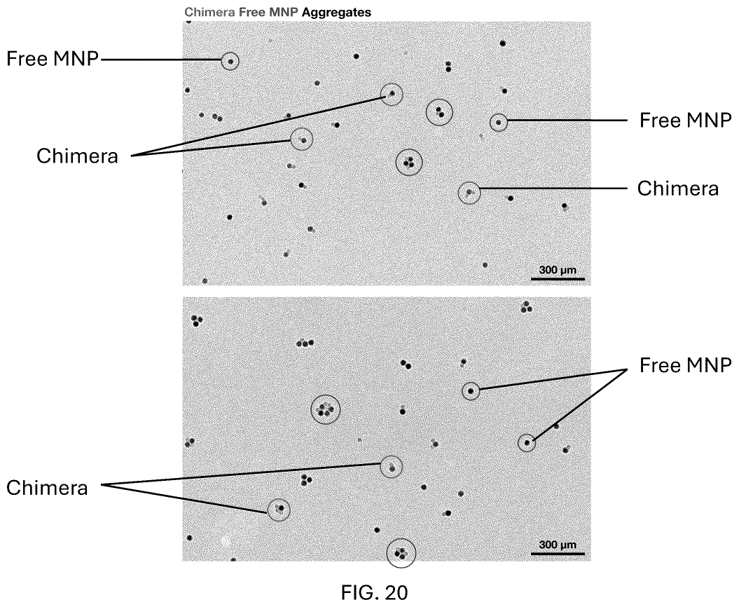

The MNP-AAV chimeras 100, 200, 300, 400, and/or 500 were then purified by centrifugation to remove non-reacted AAV-Tz (FIGS. 15A-15C). The final solution contained MNP-AAV chimeras 1200, overreacted aggregates, and non-reacted free MNPs 120 (FIG. 16). Free MNPs 120 and large aggregates may accumulate in the liver due to the lack of targeting moieties or their size, respectively. Given that additional purification (e.g. affinity chromatography) of MNP-AAV chimeras 100, 200, 300, 400, and/or 500 may decrease the total yield, the reaction conditions were instead optimized to reduce the proportion of free MNPs 120 and aggregates in the final product mixture (see FIG. 20). Increasing the Tz-NHS:Lys ratio yielded fewer free MNPs 120 and a higher average χ (χ̅) in synthesized MNP-AAV chimeras 1200 (FIG. 17-18B). However, excess of Tz groups on AAVs 110 favored aggregation and led to loss of gene carrying capacity. In contrast, adjusting the ratio of AAV 110 to MNP 120 during IEDDA conjugation allowed for the precise tuning of χ̅ without forming aggregation (FIGS. 2D and 19). At a reaction ratio of 3.3 AAVs 110 to 1 MNP 120, approximately 20% of MNPs 120 were not conjugated to an AAV 110, and >30% of MNPs 120 were in aggregates. Meanwhile, at a reaction ratio of 10 AAVs 110 to 1 MNP 120, <2% of MNPs 120 were not conjugated to an AAV 110, and <30% of MNPs 120 had aggregated.

FIG. 2B is a schematic and a TEM image of a QD chimera 101. The chimerization scheme shown in FIGS. 1C and 1D may be generalizable across nanoparticle classes and AAV 110 serotypes. For example, in addition to about 24 nm MNPs 120 cadmium selenide/zinc sulfide quantum dots 140 (QDs; about 11.5 nm in diameter, emission peak λₑₘ=650 nm) were coated with a silica (SiO₂) shell 130 and then conjugated to AAVs 110 using the chimerization scheme shown in FIGS. 1C and 1D.

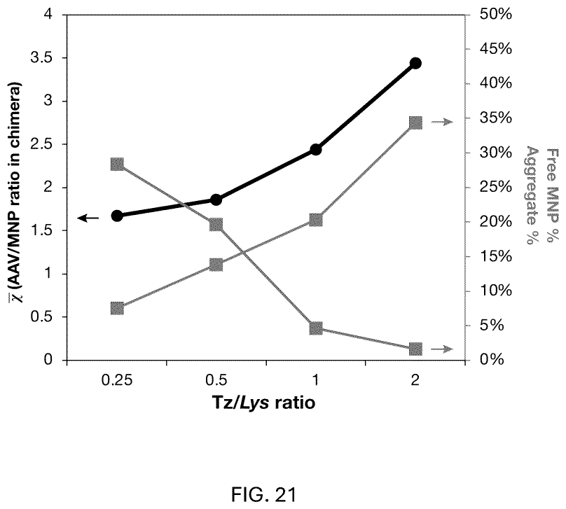

FIG. 2C illustrates MNP-AAV chimeras 100 formed from AAVs 110 with different serotypes. MNP-AAV chimeras 100 and/or QD chimeras 101 may be synthesized using various AAV 110 serotypes (see FIG. 21). For example, an MND-AAV-DJ chimera 600 (FIG. 2C, right panel) may include an AAV-DJ 610 and an MNP 620, an MNP-AAV.CAP-B10 chimera 700 (FIG. 2C, left panel) may include an AAV.CAP-B10 710 and an MNP 720, and an MNP-AAV9 chimera 800 (FIG. 2C, middle panel) may include an AAV9 810 and an MNP 820. MNPs 620, 720, and 820 may be made as described above. MNPs 620, 720, and 820 may also include a silica sell coating 130 as described above. Additional AAV 110 serotypes include, but are not limited to, AAV-LK03, AAV.MaCPNS1, and AAV.MaCPNS2. In FIG. 2C, the χ̅ dependency on the number of accessible Lys is also provided.

### AAV Tropism Governs Intracellular Delivery of MNPs Via Chimeras

AAVs 110 can guide MNPs 120 in a serotype-specific manner in vitro. Other work has shown that AAV-DJ 610 may efficiently transduce HEK293T cells, a common mammalian cell line, whereas AAV9 810 may not. Therefore, the ability of the MNP-AAV-DJ (targeting) chimera 600 and the MNP-AAV9 (non-targeting) chimera 800 to escort MNPs 620 and 820, respectively, into HEK293T cells in vitro was compared. MNPs 620 and 820 were labeled with a dye (e.g., Cy7 dye) to enable quantification of the intracellular penetration of the chimeras 600 and 800 via confocal microscopy.

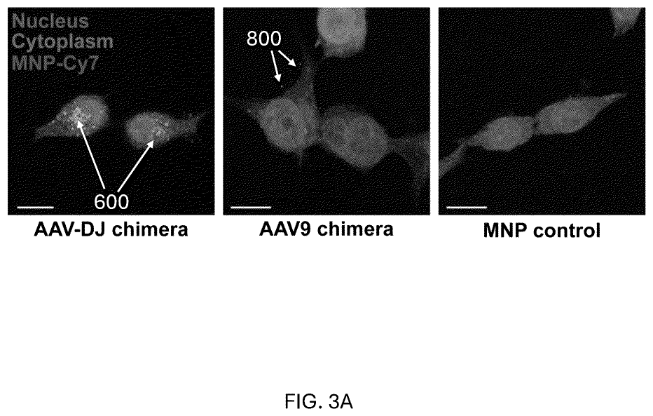

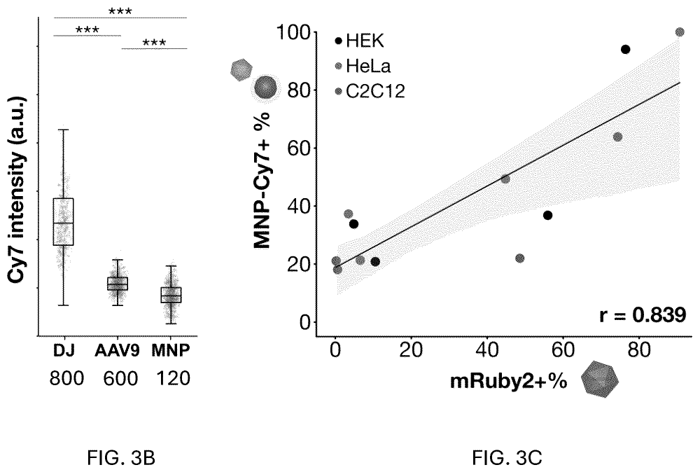

Following a 4-hour incubation with the MNP-AAV-DJ chimeras 600, bright Cy7 fluorescence was observed in the perinuclear region of HEK293T cells (FIG. 3A). In contrast, negligible MNP-Cy7 signal was detected in cells incubated with MNP-AAV9 chimeras 800 or unconjugated MNPs 120 (FIGS. 3A and 3B), indicating serotype-dependent intracellular delivery of MNPs 620 and 820 via chimeras 600 and 800, respectively.

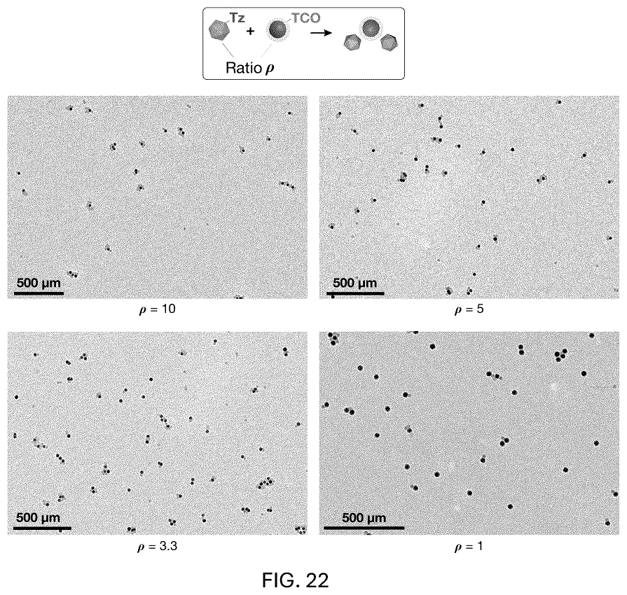

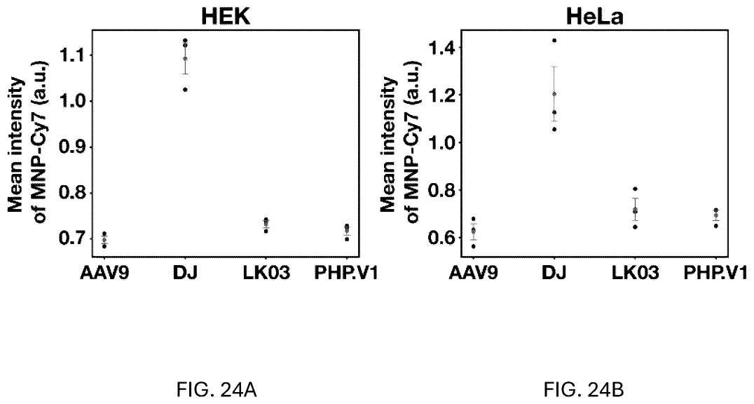

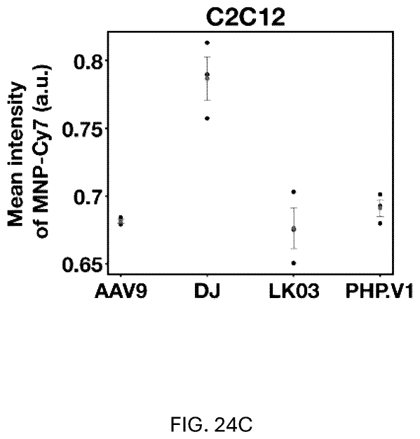

To further evaluate MNP-AAV chimera 100, 200, 300, 400, 500, 600, 700, and/or 800 targeting specificity, chimeras (χ̅=0.4−0.7) with four different AAV serotypes (AAV-DJ 610, AAV9 810, AAV.LK03 (not shown), and AAV-PHP.V1 (not shown)) were synthesized and their ability to deliver MNPs 620 and/or 820 and genes to HEK293T, HeLa, and C2C12 cell lines was evaluated. AAVs 610 and 810 packaged a gene of a red fluorescent protein, mRuby2, under a ubiquitous promoter, CAG, and MNPs 620 and/or 820 were labeled with Cy7. First, the targeting efficiency of each AAV serotype in each cell line was quantified. Following a 24-hour incubation with an AAV 610 and/or 810 or AAV.LK03 (not shown) and AAV-PHP.V1 (not shown), the percentage of mRuby2 expressing cells (mRuby2+%) was quantified via flow cytometry (see Table 2 and FIGS. 22 and 24A-24C. While AAV-DJ 610 transduced all three cell lines efficiently (>70%), AAV-LK03 transduced approximately 50% of HeLa and HEK293T (<1% of C2C12) and AAV.PHP.V1 transduced approximately 50% of C2C12 (<5% % of HEK293T and <4% % of HeLa).

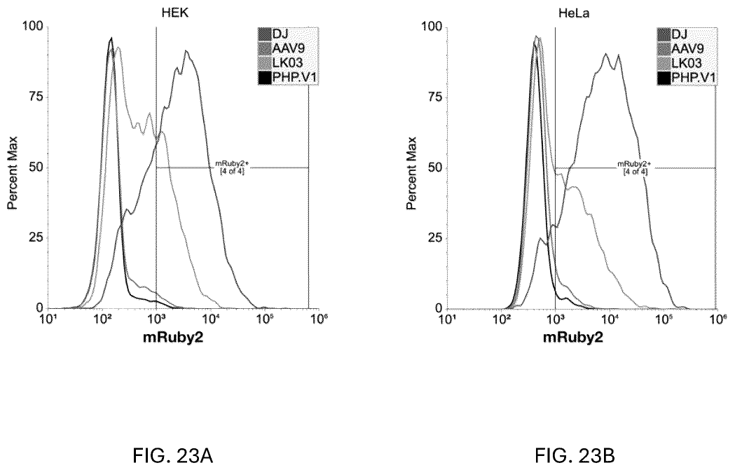

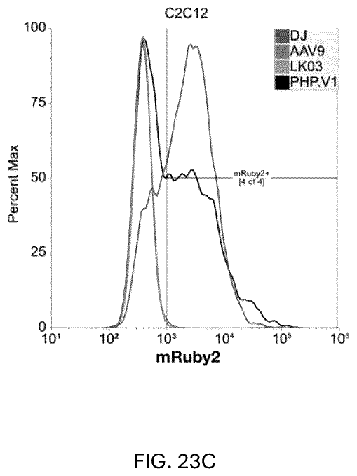

Next, these cell lines were incubated with the MNP-AAV chimeras corresponding to the four AAV serotypes (e.g., MNP-AAV chimeras 600, 800 and/or MNP-AAV chimeras with AAV.LK03 (not shown) and/or AAV-PHP.V1 (not shown)), and the percentage of MNP-Cy7 positive cells (MNP-Cy7⁺) was evaluated via confocal microscopy (Table 2 and FIGS. 23A-24C). The percentage of MNP-Cy7⁺ cells, which quantifies chimera efficacy, correlated with the AAV transduction efficacy as measured by mRuby2⁺ with a coefficient r=0.839 (FIG. 3C). The AAV serotype is included in accompanying Table 2. This observation further supports that the delivery of MNP-AAV chimeras 100, 200, 300, 400, 500, 600, 700, and/or 800 may be serotype-dependent, implying that targeting therapeutic payloads to different cell classes can be accomplished by simply swapping the AAV serotype in a chimera chemistry.

TABLE 2
mRuby2+ % and MNP-Cy7+ % for all combinations of cell lines and AAV
serotypes.

| | DJ | | AAV9 | | LK03 | | PHP.V1 | |
|---|---|---|---|---|---|---|---|---|
| | mRuby2 | Cy7 | mRuby2 | Cy7 | mRuby2 | Cy7 | mRuby2 | Cy7 |
| HEK | 76.42% | 83.3% | 10.54% | 0.5% | 55.95% | 20.6% | 4.87% | 12.2% |
| HeLa | 90.71% | 100% | 6.57% | 6.0% | 44.78% | 36.6% | 3.44% | 28.4% |
| C2C12 | 74.36% | 41.7% | 0.59% | 5.0% | 0.20% | 5.2% | 48.59% | 10.1% |

### Intracellular Fate of MNP-AAV Chimeras

Experiments in HEK293T cells revealed that following MNP-AAV-DJ chimera 600 assisted delivery, MNPs 620 labeled with Cy7 accumulated in the perinuclear region (FIG. 3A), suggesting that MNP-AAV-DJ chimeras 600 may be internalized and transported intracellularly. Live confocal imaging in HEK293T cells incubated with MNP-AAV-DJ chimeras 600 provides dynamic insight into the transport process.

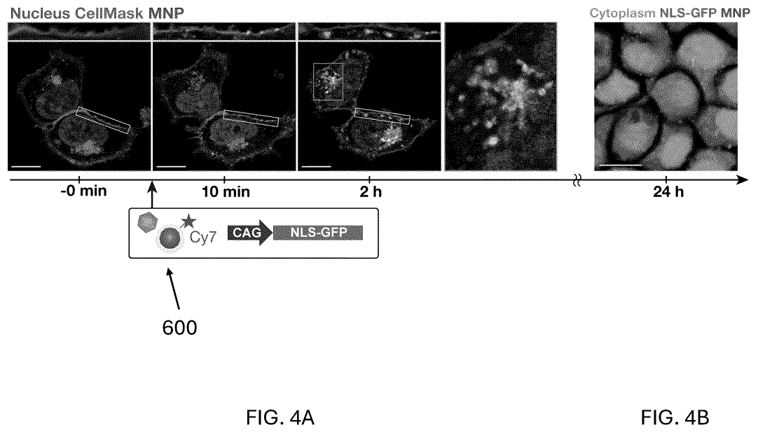

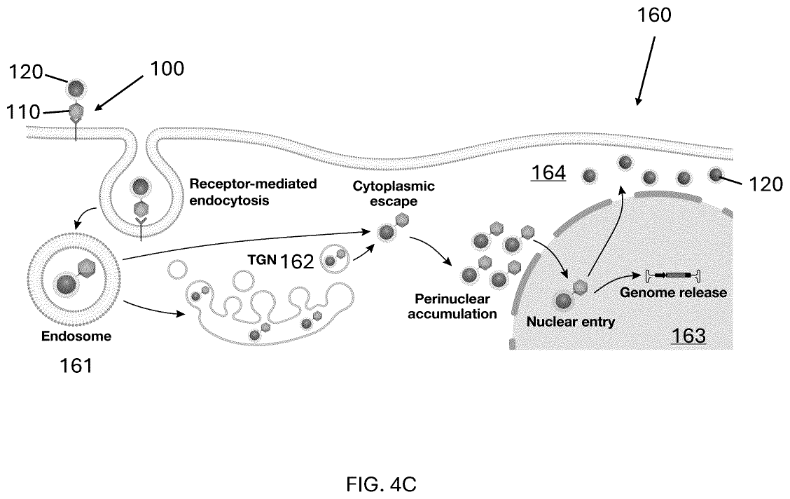

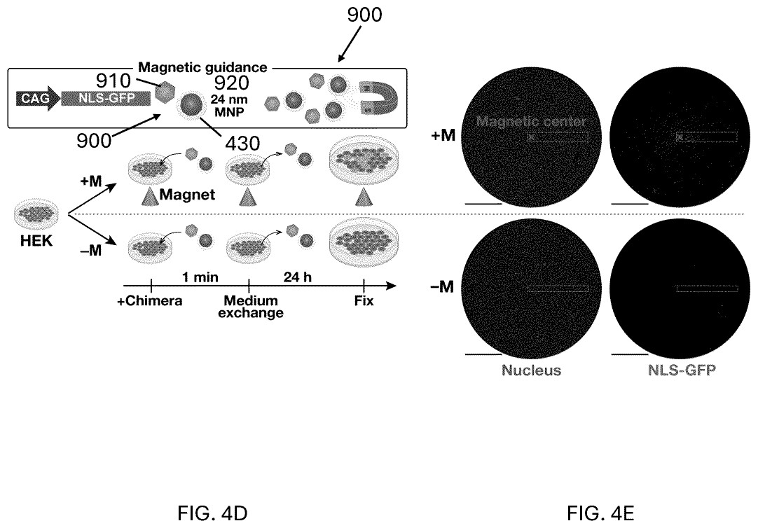

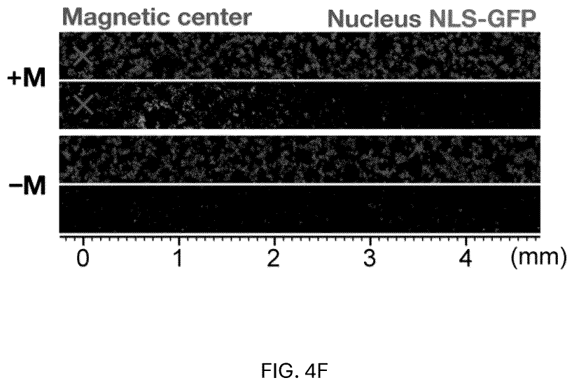

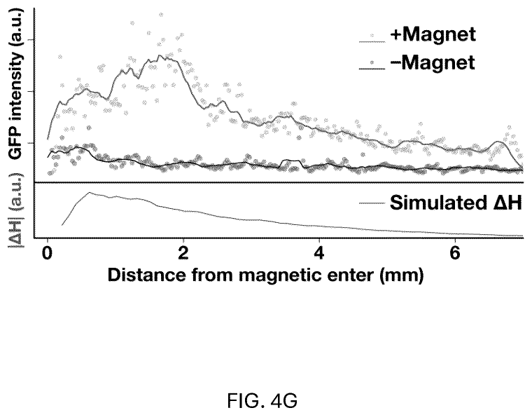

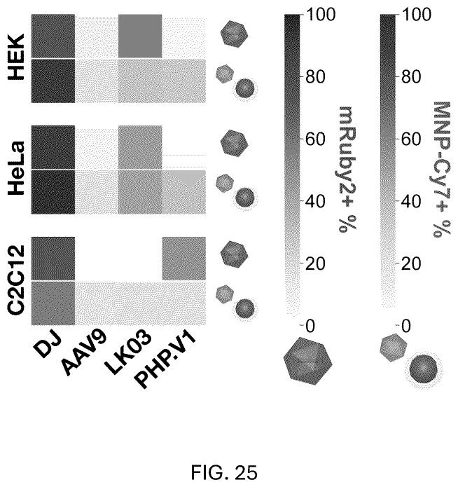

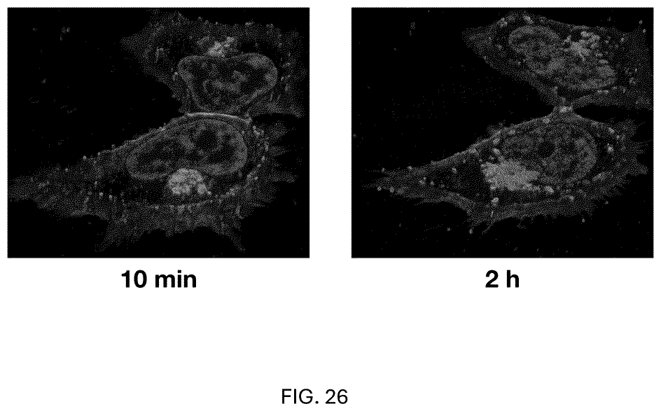

Prior to the addition of MNP-AAV-DJ chimeras 600, no Cy7 fluorescence was detected (FIG. 4A, t=−0 minutes). Ten minutes following the addition of MNP-AAV-DJ chimeras 600, MNP-Cy7 fluorescence was observed predominantly on the cell membrane (FIG. 4A, t=10 minutes and FIGS. 25-26). At 2 hours, Cy7 fluorescence on the membrane was greatly diminished, and instead Cy7 fluorescence was predominantly observed within the intracellular space (FIG. 4A, t=2 hours, and FIG. 25). Notably, at 2 hours significant intracellular colocalization between MNP-Cy7 and an amphiphilic membrane dye that also visualizes lipid bilayers inside cells, such as endosomes and the trans-Golgi network (TGN), was observed. This suggests that the MNP-AAV-DJ chimeras 600 may be internalized via endocytosis and may be transported in intracellular compartments (FIG. 4A, t=2 hours, and FIG. 26). Additionally, many MNP-AAV-DJ chimeras 600 (as marked by Cy7) accumulated in the perinuclear region, where some Cy7 clusters were surrounded by the membrane dye and some were not, suggesting MNP 620 localization within intracellular compartments and cytoplasmic escape (FIG. 4A, t=2 hours, top square box in the third panel and zoom-in image in the fourth panel). At 24 hours after MNP-AAV-DJ chimera 600 administration, the expression of a transgene (nuclear-localization signal-green fluorescent protein, NLS-GFP) packaged in the AAVs 610 was observed. This demonstrates that AAVs 610 in MNP-AAV-DJ chimeras 600 may retain their functionality as vectors and further suggests that they may enter into the nucleus (FIG. 4B).

FIG. 4C illustrates the entire proposed process of MNP-AAV chimera 100 transport. The observations disclosed herein indicate that the intracellular trafficking of MNP-AAV chimeras 100 and/or QD chimeras 101 may align with that of unfunctionalized AAVs 110. Unfunctionalized AAVs 110 may be internalized by a cell 160 by receptor-mediated endocytosis, transported to the perinuclear region via endosomes 161 and the TGN 162, and then enter the nucleus 163 to release transgenes by disassembly of their capsids. Once AAVs 110 disassemble in the nucleus, MNPs 120 bound to a virus protein monomer may be released into the cytoplasm 164 as observed at 24 hours (FIG. 4B).

### Magnetic Guidance of MNP-AAVs for Spatially Restricted Transduction

Since MNP-AAV chimeras 100 may function as viral vectors, and MNPs 120 can also be manipulated by magnetic field (MF) gradients, it was hypothesized that transgene delivery by the MNP-AAV chimeras 100 may be remotely magnetically directed. To test this hypothesis, HEK293T cells were incubated with MNP chimeras of AAV-DJ (e.g., MNP-AAV-DJ chimeras 900) carrying NLS-GFP under a CAG promoter, in the presence of magnetic field gradients for 1 minute (FIG. 4D). The MNP-AAV-DJ chimeras 900 may include an MNP 920 with a silica shell 430 as described above and AAV-DJ 910. After a medium exchange to remove MNP-AAV-DJ chimeras 900 that did not bind to their target receptors, the cells were incubated for 24 hours and allowed to express NLS-GFP. Strong expression of NLS-GFP was observed near the center of the applied MF, while weaker signals were obtained in the periphery (FIGS. 4E-4G). The fluorescence intensity peaked at approximately 1-2 mm from the magnet center tracking the simulated MF gradients (FIG. 4G, bottom curve). In the absence of a MF gradient, negligible expression of NLS-GFP was observed, indicating that the MNP-AAV-DJ chimeras 900 were not able to reach the cells prior to the medium exchange. This suggests that the MF gradient may accelerate the diffusion of MNP-AAV-DJ chimeras 900 in the medium by attracting them, an effect that increases with proximity to the magnet center. As a result, the cells near the magnet center interacted with a greater number of MNP-AAV-DJ chimeras 900, were more effectively transduced, and thus exhibited brighter NLS-GFP fluorescence.

Tissue-Specific Systemic Delivery of MNPs and Genes with MNP-AAV Chimeras In Vivo

Building on the in vitro findings, the ability of MNP-AAV chimeras 100, 200, 300, 400, 500, 600, 700, 800, and/or 900 to target specific organs following an intravenous injection in vivo governed by the AAV tropism was investigated.

### A Brain Targeting Chimera 1000

Systemic delivery of therapeutics to the brain is especially challenging due to the presence of the BBB, as well as size-dependent filtration barriers, including the liver and spleen, which selectively remove circulating nanoscale objects. MNP-AAV chimeras with AAV.CAP-B10 1010 (denoted CAP-B10 chimeras 1000) can address this challenge. AAV.CAP-B10 1010 was engineered to selectively target the brain tissue while avoiding non-specific accumulation in the liver.

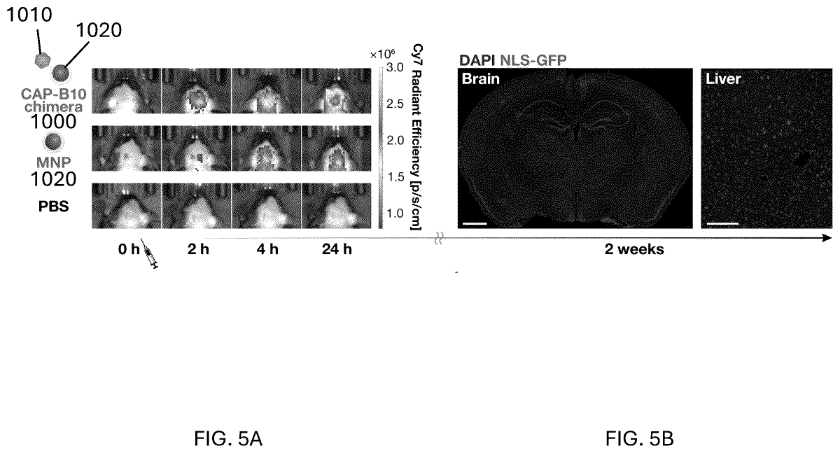

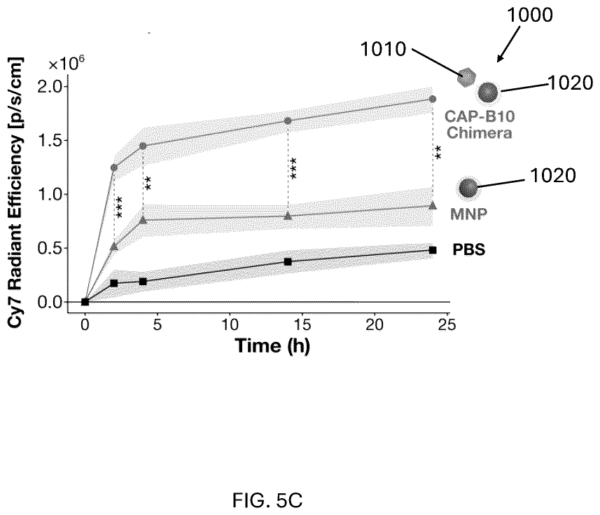

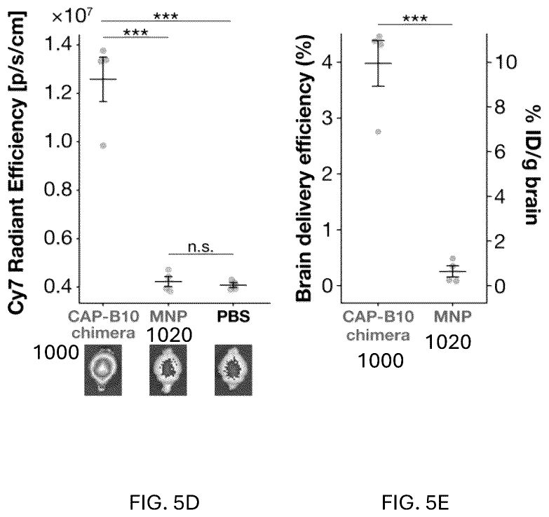

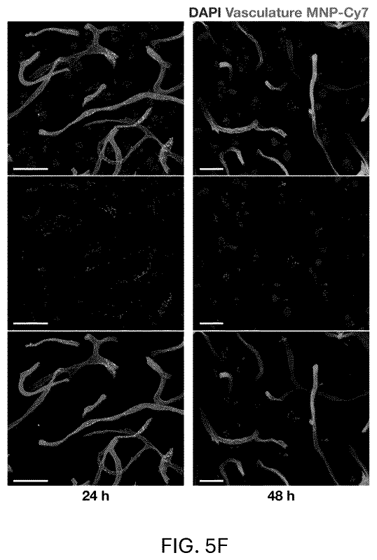

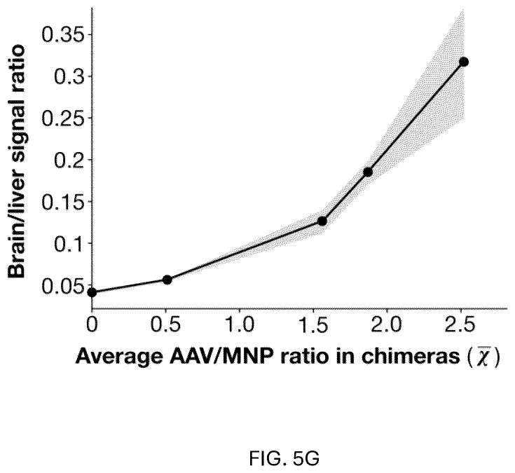

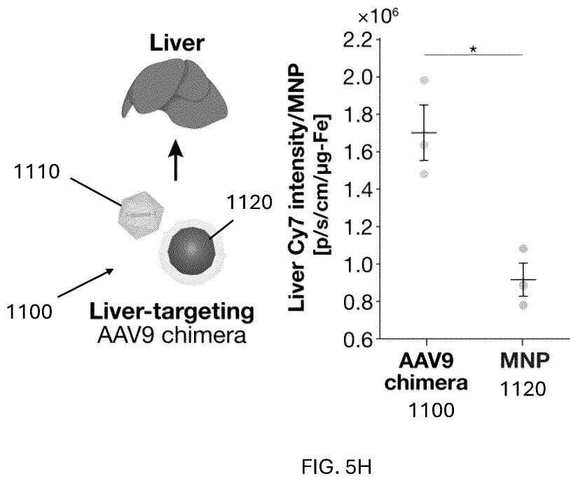

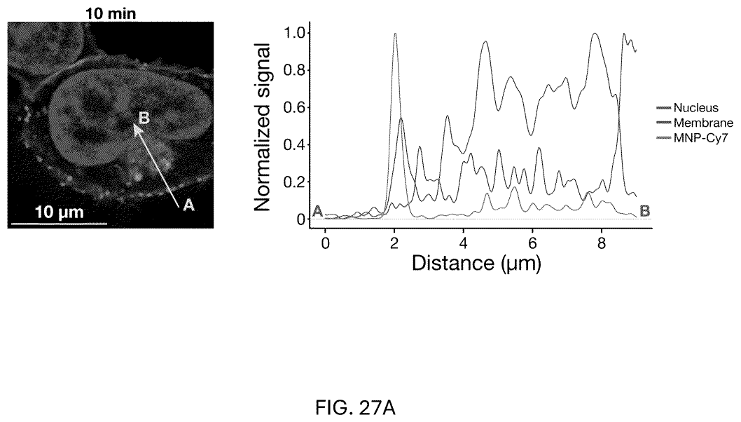

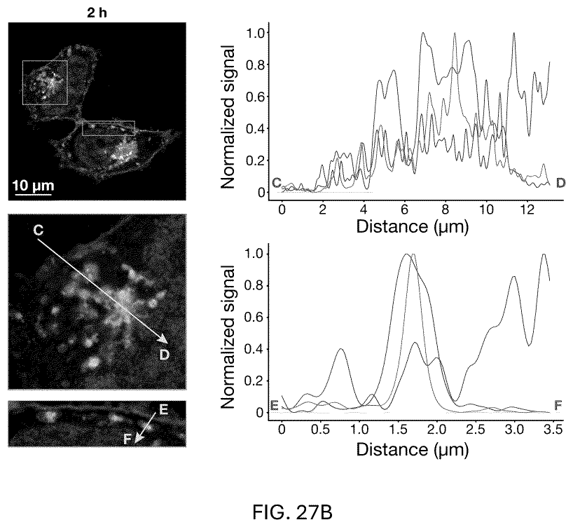

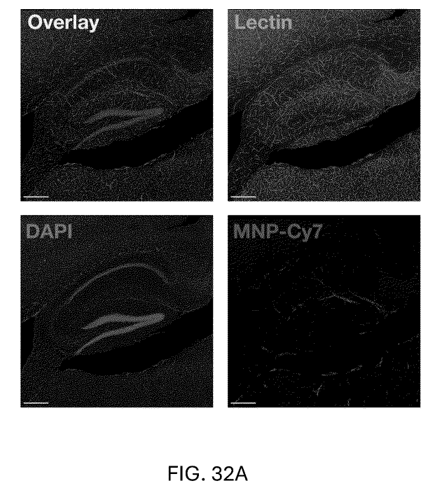

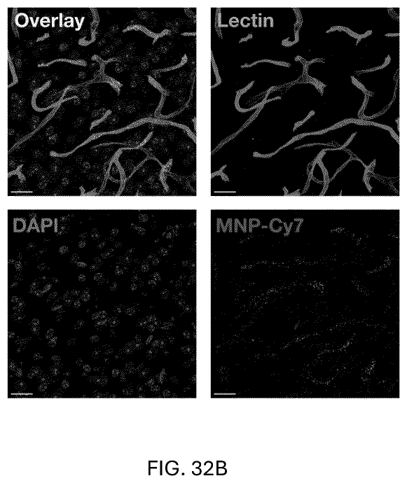

The CAP-B10 chimeras (χ̅=0.51, 100 μL, 41.2 μg_{[Fe]}/mouse), the control AAV-free MNP-Cy7 (100 μL, 41.2 μg_{[Fe]}/mouse), or phosphate-buffered saline (PBS) (100 μL) were intravenously injected through the retro-orbital route in wild-type mice (n=4 per group) (FIG. 5A). Two hours following injections, Cy7 fluorescence was detected by in vivo fluorescence imaging (IVIS) in the brain of mice injected with CAP-B10 chimeras 1000, and the signal increased at increased at 2 hours, 4 hours, 12 hours, and 24 hours post injection (FIG. 5A). Significantly lower Cy7 fluorescence was detected from the brains of control mice injected with AAV-free MNP-Cy7 1020 or PBS (FIG. 5C). The mice were perfused 24 hours after injections, and Cy7 fluorescence was quantified in the dissected brain and liver. Consistent with IVIS measurements, the signal in the brain was significantly higher in the CAP-B10 chimera 1000 group as compared to controls (FIGS. 5D, 27A, and 27B). The delivery efficiency of CAP-B10 chimeras 1000 (calculated based on Cy7 fluorescence) to the brain was found to be about 3 ID to about 20 ID, for example about 3.98±0.41% ID (9.95±1.02% ID/g brain, FIG. 5E), which is ˜approximately 10 times higher than prior reports of brain-targeting solid-state nanoparticles. Furthermore, considering the stoichiometry of the chimera solution, approximately 50% of the injected MNPs 1020 may be AAV-free, and thus the calculated delivery efficiency may be an underestimate (see FIGS. 32A and 32B). This high delivery efficiency demonstrates the potent advantages of AAVs as targeting agents.

Notably, the CAP-B10 chimeras 1000 retained their ability to deliver genes in vivo. In mice (n=3) intravenously injected with CAP-B10 chimeras 1000, packaged with CAG::NLS-GFP, bright NLS-GFP fluorescence was observed throughout the brain parenchyma 2 weeks following injection (FIG. 5B). Notably, expression of NLS-GFP was not found in the liver (FIG. 5B). These findings corroborate that the CAP-B10 chimeras 1000 and the other MNP-AAV chimeras disclosed herein may function as viral vectors with the tropism defined by the parent AAVs in vivo.

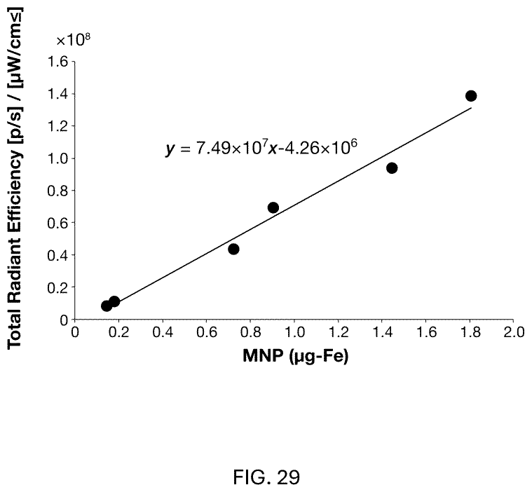

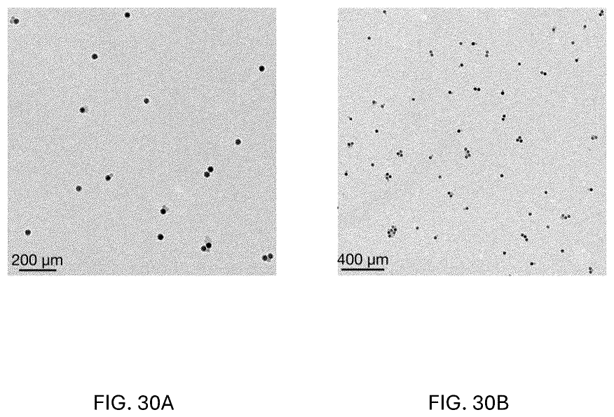

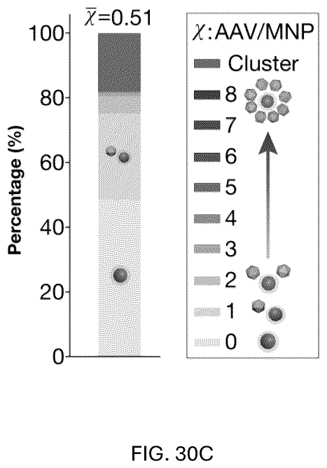

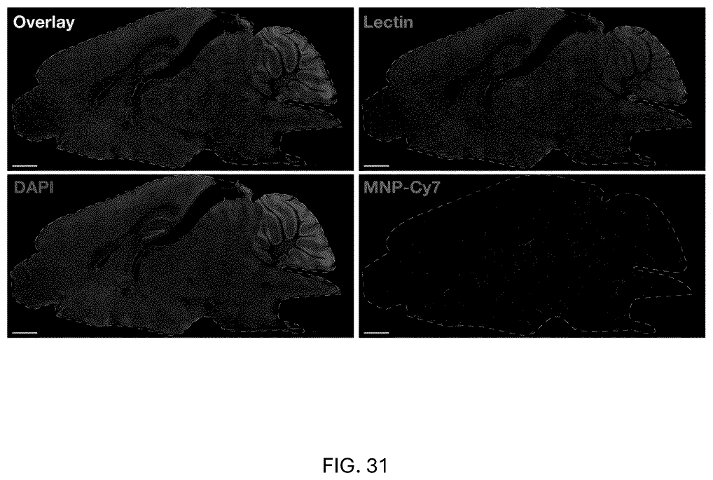

To gain further insight into MNP-AAV chimera 100 transit in vivo, the biodistribution of these particles in the brain tissue at 24 hours and 48 hours following intravenous injections (FIG. 5F) was assessed. At 24 hours post injection, MNP-Cy7 was predominantly localized within brain vasculature (FIG. 5F, ‘24 hours’ and FIGS. 29-30C)), and no detectable fluorescence was found in the parenchyma. However, at 48 hours after injection, MNP-Cy7 was detected outside the vasculature and near the nuclei of cells in the parenchyma (FIG. 5F, ‘48 hours’ and FIG. 31), suggesting that MNP-AAV chimeras 1000 may cross the BBB between 24 hours and 48 hours. This is consistent with a prior study in which AAV.PHP.eB, another brain-targeting AAV, was reported to accumulate in the vasculature at 24 hours post-injection and disappear at 48 hours.

Although CAP-B10 chimeras 1000 delivered MNPs to the brain parenchyma, MNP-Cy7 fluorescence was also detected in the liver (see FIGS. 27A and 27B). MNPs 1020 may be filtered by the liver because (1) the average AAV-to-MNP ratio (χ̅=0.51) was insufficient to achieve liver avoidance, and (2) there were many AAV-free MNPs 1020 and large aggregates in the injected solutions (see FIGS. 32A-32B).

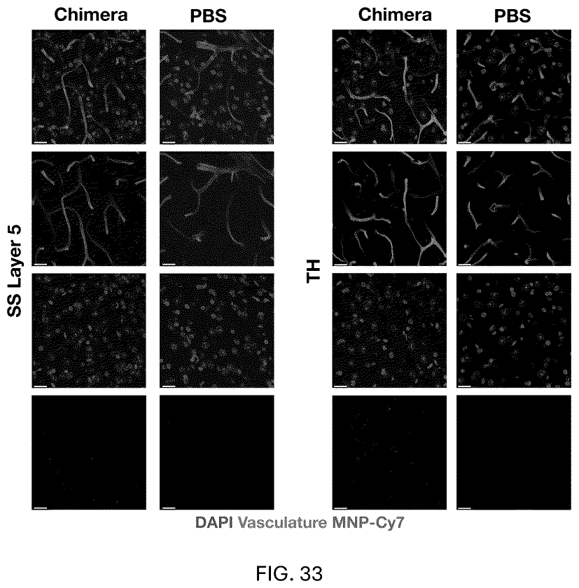

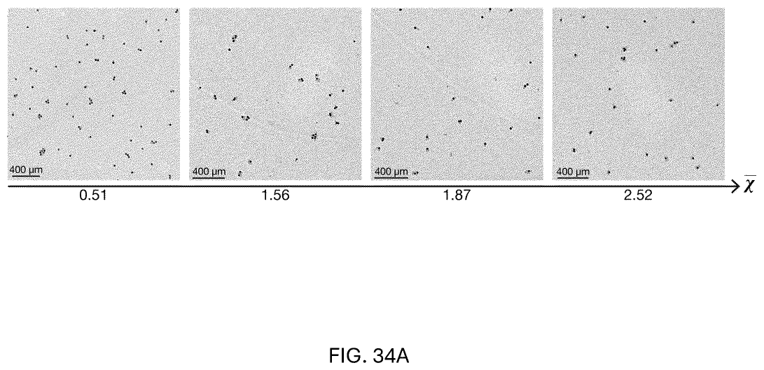

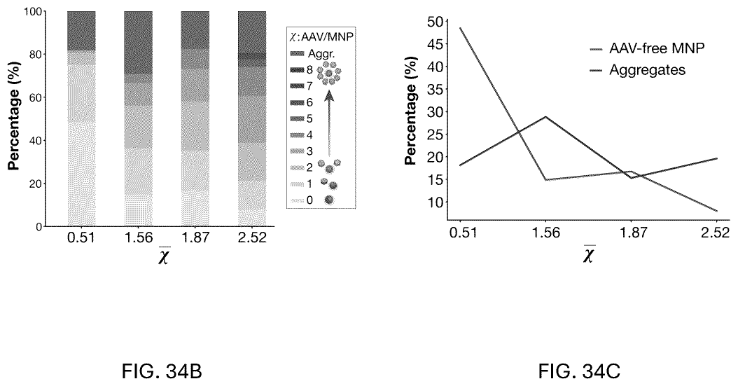

The effect of χ̅ on targeting specificity of CAP-B10 chimeras 1000 was also evaluated. FIG. 5G shows an exponential increase of the ratio of MNP-Cy7 fluorescence in the brain relative to the liver for chimeras with increasing χ̅ values −0.51, 1.56, 1.98, and 2.42 (see FIGS. 33-34C). The superior brain-targeting specificity of the chimeras with higher χ̅ may be due to a more efficient guidance afforded by the greater number of the AAV capsids 1010 linked to the MNPs 1020, and/or the decreased abundance of free MNPs 1020 and aggregates that tend to accumulate in the liver. These observations motivate future refinement of the chimera synthesis, including using a microfluidic device for a controlled synthesis scheme for increased specificity and efficacy of AAV-guided cargo delivery.

### A Liver Targeting Chimera 1100

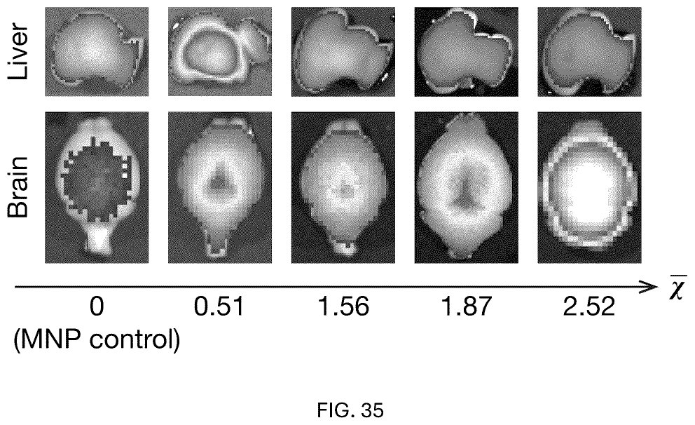

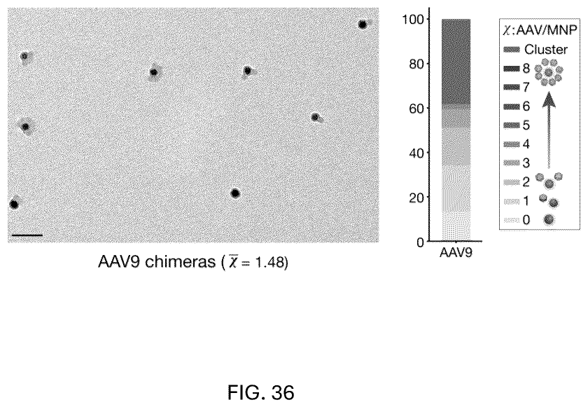

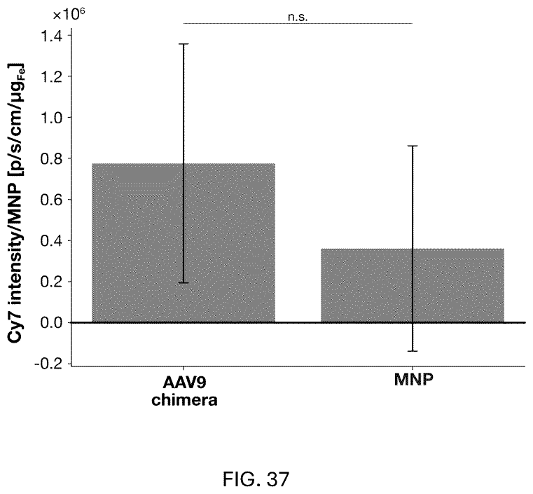

To further evaluate the role of the AAV serotype in targeting specificity MNP-AAV9 chimeras 1100 (χ̅=1.12) of MNP-Cy7 1120 and AAV9 1110, which is known to preferentially transduce the liver, was synthesized (see FIGS. 35-37). Following retro-orbital injections, MNP-AAV9 chimeras 1100 delivered MNPs 1120 to the liver efficiently where the Cy7 fluorescence was approximately 1.81 times greater than in the control group injected with MNP-Cy7 1120 particles (FIG. 5H). Notably, negligible Cy7 fluorescence was observed in the brain of these mice and no statistical difference was found between the MNP-AAV9 chimera 1100 and control groups. These findings underscore the deterministic role of the AAV serotype on the targeted delivery of the nanoscale cargo and pave way for future research in organ-specific therapeutics powered by ever-expanding array of AAVs. For example, AAV.MaCPNS2 may be used to target the peripheral nervous system (PNS) including the gut, nodose ganglia, and/or dorsal root ganglia.

### Discussion

Strategies for targeted delivery of therapeutics in vivo commonly employ small molecules, aptamers, peptides, endogenous proteins, and antibodies. However, in addition to their targeting function, these moieties may interact with a myriad of biomolecules in the blood accumulating protein coronas and prompt cargo clearance by the immune system and filtration organs. In contrast, AAVs have evolved to target specific tissues while avoiding biological barriers. Herein, these complementary AAV functions are leveraged to deliver nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770) to the brain. AAV functions may also be used to deliver nanoparticles to the brain, for example, by using a brain targeting AAV.

Iron oxide MNPs 120 were conjugated with AAVs 110 to form chimeras 100 with controlled stoichiometry through quencher-mediated IEDDA cycloaddition (FIG. 1C). The MNP-AAV chimeras 100 may deliver MNPs 120 and genes to specific cells governed by the AAV 110 serotype and its tropism (FIGS. 3A-3C). The MNP-AAV chimeras 100 additionally may permit magnetic localization of gene delivery in vitro, paving the way to spatially-restricted transduction with broadly administered vectors (FIGS. 4D-4G).

By leveraging a brain-targeting AAV serotype (e.g., AAV.CAP-B10 1010) CAP-B10 chimeras 1000 may deliver MNPs 1020 to the brain parenchyma in mice following an intravenous injection (FIGS. 5A-5E). The delivery efficiency of CAP-B10 chimeras 1000 was approximately 4% ID (approximately 10% ID/g-brain), which is approximately 10 times higher than previous strategies for transporting nanoparticles to the brain. These findings illustrate that the targeting and the ability to escape clearance characteristic of AAVs 110, 210, 310, 410, 510, 610, 710, 810, 910, 1010, 1110, 1210, and/or 1310 can be conferred to other materials (e.g., MNPs 120, 220, 320, 420, 520, 620, 720, 820, 920, 1020, and/or 1120) through direct conjugation. This approach may be generalizable across nanoparticle classes as illustrated by the synthesis of the MNP-AAV chimeras 100, 200, 300, 400, 500, 600, 700, 800, 900, 1000, 1100, 1200, and/or 1300 and QD-AAV chimeras 101 (FIGS. 2A and 2B and can be extended to molecular payloads as shown with AAV-Tz (Fig. S3).

The specificity of MNP 1020 targeting to the brain may be improved by increasing the ratio of AAV.CAP-B10 1010 within the chimeras 1000 (FIG. 5G), consistent with the brain-targeting and efficient liver avoidance of this serotype. These findings suggest that multi-component capsid structures may help to realize the disparate properties of targeting a specific tissue and “de-targeting” other organs. Notably, these findings motivate future exploration of synthetic moieties (e.g., a virus-mimicking artificial protein) that recapitulate the targeting and avoidance properties of the capsids while being amenable to inexpensive cell-free synthesis. The design of such moieties demands further mechanistic insight into biophysical interactions of AAVs, such as AAVs 110, 210, 310, 410, 510, 610, 710, 810, 910, 1010, 1110, 1210, and/or 1310, with various tissues and may additionally benefit from emerging machine-learning approaches. However, the scheme reported herein enables AAV functionalization with approximately 60-80% efficiency. Additionally, the rapidly growing palette of AAV serotypes may motivate the use of the MNP-AAV chimeras 100, 200, 300, 400, 500, 600, 700, 800, 900, 1000, 1100, 1200, and/or 1300 and/or the QD chimeras 101 disclosed herein as a novel class of tissue-specific and non-immunogenic shuttles of therapeutic payloads to a variety of organs following systemic intravenous delivery.

### Methods

### Reagents

Iron chloride hexahydrate (98-102%, #44944), 1-octadecene (ODE; 90%, #O806), benzyl ether (BE; 98%, #108014), oleic acid (OAc; 90%, #364525), Igepal CO-520 (average Mn=441, #228643), ammonium hydroxide (NH₄OH; 28.0-30.0%, #221228), tetraethyl orthosilicate (TEOS; 99.999%, #333859), [3-(2-aminoethylamino) propyl]trimethoxysilane (AEAPTMS; ≥80%, #440302), tetramethylammonium hydroxide (TMAOH; 25 wt % in methanol, #334901), CdSe/ZnS core-shell type quantum dots (QDs; dem 650 nm, #919136), and trioctylamine (98%, #T81000) were purchased from Sigma-Aldrich. Sodium oleate (>97%, #O0057) was purchased from TCI Chemicals. Cy5-Tetrazine (Tz) (#130E0) and Sulfo-Cy7-Tz (#153E0) were purchased from Lumiprobe. Methoxy poly(ethylene glycol) 24-NHS (mPEG-NHS; #BP-23970), trans-cyclooctene-PEG24-NHS (TCO-PEG24-NHS; #BP23970), Tz-PEG5-NHS ester (#BP-22681), mPEG-methyltetrazine (mPEG5k-Tz) (Mw 5000, #BP-26353), and mPEG4-TCO (BP-27872) were purchased from BroadPharm. General solvents were purchased from Fisher Scientific. All chemicals were used without further purification.

### Plasmids

The MNP-AAV chimeras 100, 200, 300, 400, 500, 600, 700, 800, 900, 1000, 1100, 1200, and/or 1300 and/or the QD-AAV chimeras 101 disclosed herein may include, but are not limited to, the following AAV plasmids: pUCmini-iCAP-AAV.CAP-B10 (Addgene plasmid #175004; http://n2t.net/addgene: 175004; RRID:Addgene_175004), pUCmini-iCAP-PHP.V1 (Addgene plasmid #127847; http://n2t.net/addgene: 127847; RRID:Addgene_127847), pUCmini-iCAP-AAV.MaCPNS1 (Addgene plasmid #185136; http://n2t.net/addgene: 185136; RRID:Addgene_185136), pUCmini-iCAP-AAV.MaCPNS2 (Addgene plasmid #185137; http://n2t.net/addgene: 185137; RRID:Addgene_185137), pAAV-CAG-mRuby2 (Addgene plasmid #99123; http://n2t.net/addgene: 99123; RRID:Addgene_99123), pAAV-CAG-mNeonGreen (Addgene plasmid #99134; http://n2t.net/addgene: 99134; RRID:Addgene_99134), and CAG-NLS-GFP (Addgene plasmid #104061; http://n2t.net/addgene: 104061; RRID:Addgene_104061) were used herein. pAAV2/9n was also used herein (Addgene plasmid #112865; http://n2t.net/addgene: 112865; RRID:Addgene_112865). Additionally, AAV-LK03 was also used herein (Addgene plasmid #206512; http://n2t.net/addgene: 206512; RRID:Addgene_206512). AAV-CAG-jGCaMP8s-WPRE were also used herein (Addgene plasmid #179256; http://n2t.net/addgene: 179256; RRID:Addgene_179256). pAAV-DJ (Cell Biolabs, Inc. #VPK-420-DJ) and pHelper were purchased from CELL BIOLABS, INC.

### Magnetic Nanoparticle Synthesis

Magnetic nanoparticles (MNPs) 120, 220, 320, 420, 520, 620, 720, 820, 920, 1020, and/or 1120 of Fe₃O₄ (magnetite) may be synthesized by the thermal decomposition method, using iron oleate synthesized from sodium oleate and FeCl₃·6H₂O as a precursor. Approximately 37.7 g (123 mmol) of sodium oleate and 10.81 g (40 mmol) of FeCl₃·6H₂O were placed in a 250 mL three-neck flask with a mixture of 100 mL of hexane, 50 mL of ethanol, and 50 mL of Milli-Q water, and heated to 70° C. for 90 minutes under N₂. The resulting black liquid containing iron oleate was washed 5 times with Milli-Q water in a separatory funnel, and then dried at 110° C. on a Schlenk line overnight. The dried iron oleate is a black, viscous solution, which can be stored under vacuum for up to one year.

For MNP 120, 220, 320, 420, 520, 620, 720, 820, 920, 1020, and/or 1120 synthesis, approximately 2.7 g (3 mmol) of iron oleate was placed in a 250 mL three-neck flask and mixed with 6 mL of 1-octadecene, 3 mL of benzyl ether, and 1.92 mL of oleic acid. To control the size of MNPs 120, 220, 320, 420, 520, 620, 720, 820, 920, 1020, and/or 1120, the amount of oleic acid was adjusted. For example, for 24 nm MNPs, about 5 mmol to about 7 mmol, for example, about 6 mmol of oleic acid may be added. The solution was degassed at 90° C. under vacuum for 30 minutes while stirring at 100 rpm. The flask was then heated to reflux at 330° C. under N₂. After reacting for 30 minutes, the solution was cooled down to room temperature, and the MNPs 120, 220, 320, 420, 520, 620, 720, 820, 920, 1020, and/or 1120 were washed with 80 mL of hexane by centrifugation at 8,000 g for 10 minutes at room temperature. The resulting pellet was then washed three times with a mixture of ethanol and hexane (ethanol:hexane=1:4 (volume ratio)) at the same centrifugation conditions. The washed MNPs 120, 220, 320, 420, 520, 620, 720, 820, 920, 1020, and/or 1120 were then resuspended in 3 mL of chloroform and stored at 4° C. The concentration of Fe was measured by inductively coupled plasma atomic emission spectroscopy (ICP-AES). The size of MNPs 120, 220, 320, 420, 520, 620, 720, 820, 920, 1020, and/or 1120 was determined by TEM image analysis on a Fiji/ImageJ. Based on this analysis, the numerical density of MNPs 120, 220, 320, 420, 520, 620, 720, 820, 920, 1020, and/or 1120 were calculated.

### MND Synthesis

Magnetic nanodiscs (MNDs) MNDs 670 may be synthesized according to Gregurec et al., “Magnetic Vortex Nanodiscs Enable Remote Magnetomechanical Neural Stimulation,” ACS Nano 2020 14 (7), 8036-8045. Non-magnetic hematite nanodiscs (NDs) were synthesized using the hydrothermal method. Approximately 800 mg of sodium acetate and 273 mg of FeCl₃·6H₂O were placed in a Teflon vessel. After 10 mL of ethanol and 800 μL of DI water were added, the Teflon vessel was sealed tightly and heated at 180° C. for 18 hours. The synthesized red solution was washed with ethanol 3 times by centrifuge, and then the pellet of hematite NDs was resuspended in 5 mL of ethanol.

Approximately 100 mg of the hematite NDs were placed in a 250-mL three-neck flask with 2.22 mL of oleic acid and 29 mL of trioctylamine. The solution was heated at 370° C. with H₂ bubbling. Heating was stopped once the color of the solution changed from red to black. The black magnetite NDs (MNDs) 670 were washed with a 1:1 mixture of hexane and ethanol two times and with chloroform three times using magnetic separation. The MNDs 670 were resuspended in 1 mL of chloroform and stored at 4° C.

### Silica Coating and Amine Functionalization

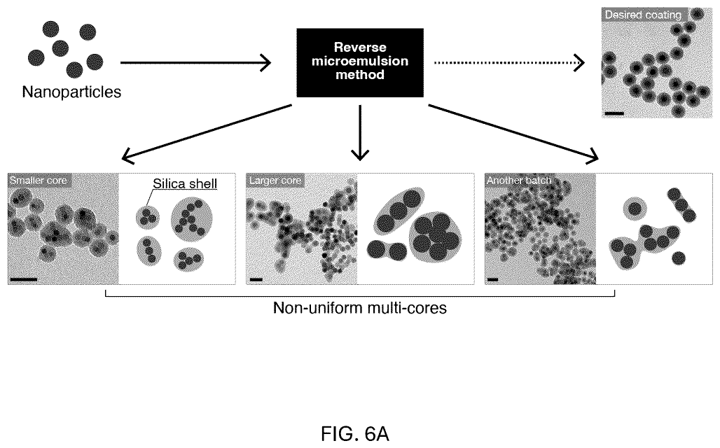

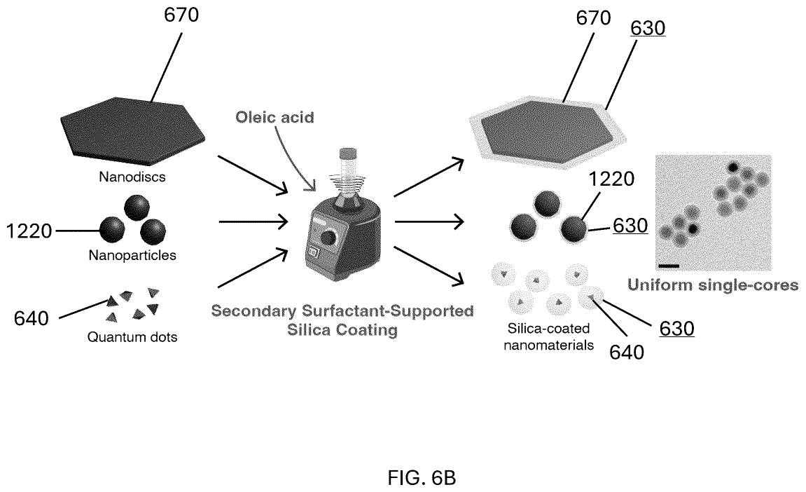

Chemical interactions between nanoparticles and their surroundings are governed by the composition of molecules or functional groups at their surfaces. However, the surfaces of nanoparticles are diverse and complex, and it can therefore be challenging to specifically tailor a nanoparticle's surface coating to enable its use in an intended application. A universally applicable method to produce a standard nanoparticle surface coating on a range of nanoparticle compositions would therefore greatly benefit multiple areas of nanotechnology research. Thin silica shell coatings can achieve this goal but creating uniform and reproducible coatings is not always straightforward. Common issues encountered in these methods include aggregation and fusion of silica shells during their growth, uneven shell thickness, and inconsistent or non-reproducible protocols (see FIG. 6A).

Furthermore, Polymer coatings may be used to phase-transfer as-synthesized oleic acid-capped MNPs to polar solvent (e.g., water), where polymers were anchored to the surface of MNPs via hydrophobic interaction with oleic acid molecules. However, these polymer coatings may be disrupted in blood after intravenous injection due to their interactions with plasma proteins.

A silica shell coating (e.g., coating 130 and/or 630) circumvents these challenges by modifying the standard reverse microemulsion coating method to ensure all nanoparticle surface coatings are uniform prior to silica deposition. By adding oleic acid to the coating solution as a supplementary surfactant, various nanoparticles may be stabilized throughout the coating process. The methods disclosed herein may ensure uniform coatings while also preventing aggregation or fusion of the silica shells 130 and/or 630. Additionally, the methods disclosed herein may be highly reproducible and broadly applicable to a range of nanoparticle compositions, sizes, and shapes without the need to re-optimize the protocol. For example, as illustrated in FIG. 6B, the silica shell coatings 630 may be applied to MND 670, MNP 1220, and/or QDs 640. The silica shell coatings 130 and/or 630 may also be applied to MNPs 120, 220, 320, 420, 520, 620, 720, 820, 920, 1020, and/or 1120 and/or QDs 140 as described above. Silica-coated nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770) produced by the methods disclosed herein can be modified with diverse ligands, like polyethylene glycol, and/or targeting molecules (e.g., AAVs 110, 210, 310, 410, 510, 610, 710, 810, 910, 1010, 1110, 1210, and/or 1310), with additional chemistry. The methods disclosed herein therefore present a straightforward, universal approach to produce uniform nanoparticle surface coatings for a broad range of material applications.

Controlling the properties of nanoparticle surfaces, which determine the interactions between nanoparticles and their surroundings is vital when nanoparticles are applied to biological settings. A nanoparticle with promising physical properties may be toxic without proper surface treatment. Moreover, nanoparticles used for therapeutic applications often should be modified with chemical functionalities that target specific cells, molecules, and/or biologics.

The encapsulation of these nanoparticles with a thin silica shell (e.g., coating 130 and/or 630) may present a solution for surface modification, since silica is biocompatible and can be readily modified with multiple types of chemical reactions. Although several silica coating methods have been developed, the reverse microemulsion method (RMM) is the primary method used for nanoparticles with hydrophobic surfaces. Since many colloidal nanoparticles can be obtained with hydrophobic surface ligands, like oleylamine or oleic acid (OAc), RMM offers a method that can be applied to a wide range of colloidal nanoparticles.

Nevertheless, RMM has drawbacks that limit its use in different applications. A notable challenge is the tendency of nanoparticles to aggregate during silica shell formation, which affects the size of the final particles and can also alter the properties of the core nanoparticle as illustrated in FIG. 6A. Different methods to obtain uniformly coated nanoparticles have been reported, but these methods may not be able to be replicated. One source of this lack of reproducibility comes from the large variations in the initial surface state of the particles being coated. For example, the aggregation of core materials occurs more readily when the nanoparticles surfaces are not completely capped with surfactant molecules. Because these ligands are only weakly bound to the nanoparticle surface, they can be unintentionally lost during purification, storage, or preparation steps prior to silica embedding. Adulteration of the particles' surfactant coatings results in inconsistent surface states and thereby makes silica shell coating protocols less reproducible.

A robust method, termed S⁴C (Secondary Surfactant-Supported Silica Coating) consistently produces uniform silica coatings (e.g., coating 130) on nanoparticles (e.g., MNPs 120, 220, 320, 420, 520, 620, 720, 820, 920, 1020, 1120 and/or 1220, QDs 140 and/or 640, and/or MND 670). In the S⁴C method, the formation of multi-core particles is prevented by adding oleic acid (OAc) to the coating solution. The addition of this excess surfactant may enable homogeneous, single-core silica shell coatings 130 on nanoparticles (e.g., MNPs 120, 220, 320, 420, 520, 620, 720, 820, 920, 1020, 1120 and/or 1220, QDs 140 and/or 640, and/or MND 670) and may improve reproducibility of the silica embedding protocol. Additionally, the S⁴C method may even produce thin silica shells (e.g., <about 1 nm, for example about 0.7 nm to about 1 nm), which is challenging for other RMM methods due to aggregation of the core nanoparticles. Furthermore, the S⁴C method can be applied to a variety of sizes and compositions of nanoparticles (e.g., MNPs 120, 220, 320, 420, 520, 620, 720, 820, 920, 1020, 1120 and/or 1220, QDs 140 and/or 640, and/or MND 670) and can also be subsequently modified with standard silica-functionalization chemistries to attach a range of different surface molecules (e.g., ligands, like polyethylene glycol, amines, fluorescent dyes, and/or targeting molecules, including but not limited to, AAVs 110, 210, 310, 410, 510, 610, 710, 810, 910, 1010, 1110, 1210, and/or 1310). The S⁴C method therefore may provide a simple, reproducible, and universal method for silica shell coating 130 of the nanoparticles disclosed herein.

In the S⁴C method the as-synthesized oleic acid-capped MNPs (e.g., MNPs 120, 220, 320, 420, 520, 620, 720, 820, 920, 1020, 1120, and/or 1220) may be coated with SiO₂ by silica coating with hydrophobic ligands (e.g., Oleic acid or oleylamin) in reverse microemulsion method. Approximately 25 mL of cyclohexane was placed in a 50-mL falcon tube. The silica shell may be about 0.7 nm to about 20 nm, about 2 nm to about 8 nm thick, preferably about 4 nm thick. For the standard thickness of the silica shell (approximately 4 nm), 250 μL of oleic acid and 1540 mg of Igepal CO-520 were added and vigorously mixed. To control the thickness of the silica shell, the amount of TEOS may be adjusted. For example, for a 4 nm-thick silica shell, about 4 μL of TEOS was added, followed by vortexing for 48 hours. Alternatively, for a thicker silica shell (e.g., a silica shell >4 nm-thick), greater than 4 μL of TEOS may be used. Then, 900 pmol of MNP 120, 220, 320, 420, 520, 620, 720, 820, 920, 1020, and/or 1120 in chloroform (e.g., about 40-80 μL) was added and mixed well by vortexing. Next, 210 μL of NH₄OH was added to the solution and mixed immediately. After adding 4 μL of tetraethyl orthosilicate (TEOS), the tube was vortexed for 48 hours at room temperature. To functionalize the silica shell 130 and/or 630 with amines, approximately 1 μL of [3-(2-aminoethylamino) propyl]trimethoxysilane (AEAPTMS) was added to the same solution and vortexed another 90 minutes.

To stop the reaction and purify the amine-functionalized of the MNPs 120, 220, 320, 420, 520, 620, 720, 820, 920, 1020, 1120, and/or 1220 with the silica coating 130 and/or 630, also referred to as silica coated MNPs or MNP@SiO₂—NH₂, 4 mL of 50 mM tetramethylammonium hydroxide (TMAOH) in methanol was added. The tube was shaken for 5 seconds and let stand for 30 seconds to allow the phase separation between methanol and cyclohexane. The black bottom layer was collected in another 50-mL falcon tube and spun in a centrifuge at 10,000 g for 10 minutes. The pellet was resuspended in the same TMAOH solution and spun at the same condition. The pellet was resuspended in 4 mL of dimethyl sulfoxide (DMSO) and sonicated, then centrifuged at 20,000 g for 20 minutes at room temperature. The final pellet was resuspended in 400 μL of DMSO and stored at room temperature. The thickness of silica layer 130 and/or 630 was determined based on TEM images and the concentration of MNP@SiO₂—NH₂ was determined by ICP-AES.

QDs 140, 640, and/or 740 may also be coated using the SAC method described above. QDs 140, 640, and/or 740 were purchased from Sigma-Aldrich (#919136, 11 nm, 5 mg/mL). For silica shell coating of QDs 140, 640, and/or 740, the same recipe as described above except for the amount of QDs and TEOS. Typically, about 100 μL of QD solution at 5 mg/mL in toluene may be added to the reaction solution and to obtain approximately 32 nm silica-coated QDs 140, 640, and/or 740 and about 8 μL of TEOS may be added, for example.

MNDs 670 may also be coated using the S⁴C method described above. The recipe for MNDs 670 is similar to the S⁴C method described above except for the amount of MNDs 670 and TEOS. About 2 mg of MNDs 670 in chloroform was added to the reaction solution. For example, about 20 μL of TEOS was added to obtain approximately 4 nm-thick silica shells.

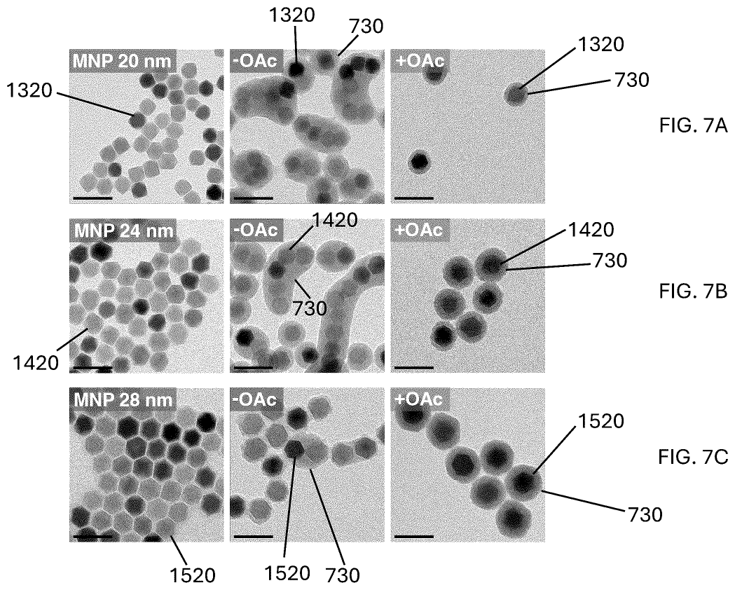

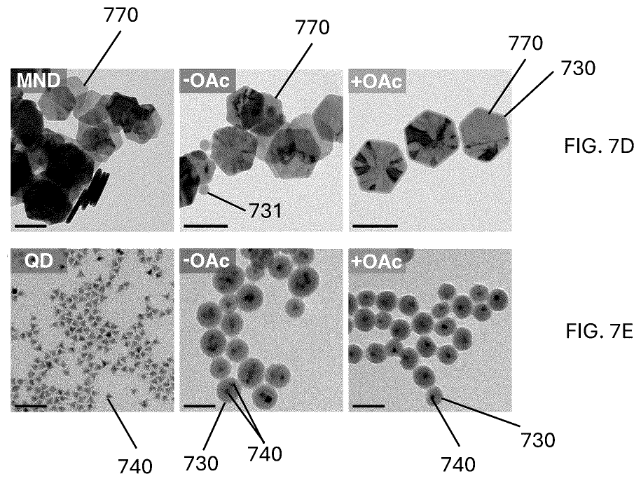

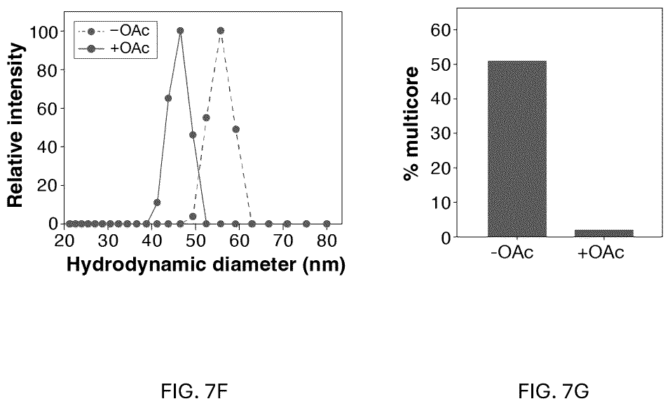

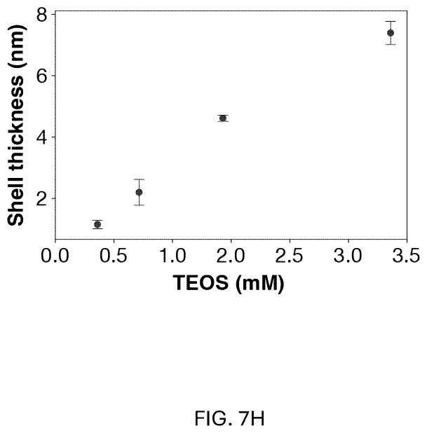

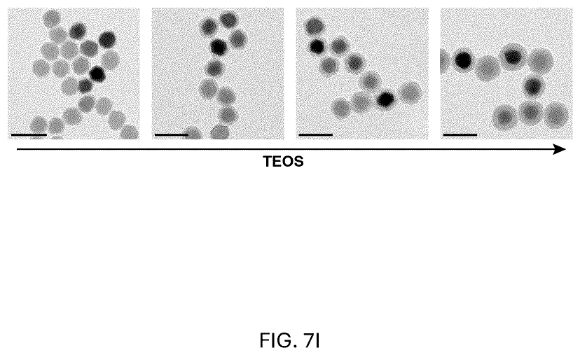

OAc is commonly used as a surfactant for nanoparticles to preserve their colloidal stability and prevent nanoparticle agglomeration. OAc-capped nanoparticles (e.g., MNPs 120, 220, 320, 420, 520, 620, 720, 820, 920, 1020, 1120 and/or 1220, QDs 140 and/or 640, and/or MND 670) may aggregate during the RMM silica shell formation reaction when their surfaces are not fully capped with OAc (due to particle storage or washing steps between their synthesis and coating). Introducing excess OAc into the silica shell coating solution may therefore be expected to stabilize individual particles and prevent the formation of multi-core particles by ensuring that all particle surfaces were uniformly capped with OAc. To test this hypothesis, the standard RMM technique was used to silica-coat spherical Fe₃O₄ magnetic nanoparticles (MNPs) of 20 nm, 24 nm, and 28 nm in diameter, MNPs 1320, 1420, and 1520, respectively. MNPs 1320, 1420, and 1520 were synthesized via a protocol that capped the particles with OAc (FIGS. 7A-7C). When these MNPs 1320, 1420, and 1520 were directly incorporated into typical RMM methods, the final samples included of approximately >80% multi-core particles with a silica shell 730 with a non-uniform shell thickness (FIGS. 7A-7C, middle panels labeled with ‘−OAc’). In contrast, when the same protocol and precursor materials were used, but excess oleic acid was added to the coating solution, the coating method produced almost exclusively single-core particles with a silica shell 730 with uniform shell thicknesses (FIGS. 7A-7C, right panels labeled with ‘+OAc’). This result was confirmed both with hydrodynamic diameter measurements using dynamic light scattering (DLS) and TEM imaging (FIGS. 7A-7F). These outcomes were consistent across different batches of MNPs and could be independently reproduced. These data therefore suggest that addition of excess oleic acid may effectively stabilize individual MNPs during the RMM method, thereby preventing agglomeration or uneven silica shell deposition.

To determine if this modification of the RMM process could be more broadly applied, the S⁴C method was used to coat larger Fe₃O₄ magnetic nanodiscs (MNDs 770), about 200 nm in diameter and about 30 nm thick (FIG. 7D). Notably, the standard RMM protocol without added OAc was ineffective for MNDs, resulting in uncoated MNDs and the formation of small silica spheres 731 (FIG. 2D, middle panel labeled with ‘−OAc’). Conversely, when oleic acid was added, MNDs 770 uniformly coated with silica shells 730 (FIG. 2D, right panel labeled with ‘+OAc’). Without being bound by any particular theory, it is hypothesized that this result could occur because the OAc may enhance the colloidal stability of the larger MNDs, allowing discrete MNDs 770 to enter the reverse micelles where the silica condensation reaction occurs. Without oleic acid, the MNDs 770 instead aggregate, and these aggregates are likely too big to enter the reverse micelles, preventing their coating by the conventional method.

Both MNPs 1220, 1320, 1420, 1520, and MNDs 670, 770 are ferrite (Fe₃O₄), but it was anticipated that the S⁴C method may also be compatible with other nanoparticles capped with OAc, as well. Indeed, oleic acid-capped CdSe/ZnS quantum dots 740 (QDs) (d=11.5 nm, λₑₘ=650 nm) subjected to the S⁴C protocol were also coated with silica (FIG. 7E), resulting in uniformly coated single-core particles (FIG. 7E, right panel labeled with ‘+OAc’). In contrast, ss with the ferrite nanoparticles, RMM without oleic acid resulted in mostly multicore particles with multiple QDs 740 coated in a single silica shell 730 (FIG. 7E, middle panel labeled with ‘−OAc’). (see also FIG. 7G). The S⁴C method may therefore be applied to various oleic acid-capped nanoparticles (e.g., MNPs 120, 220, 320, 420, 520, 620, 720, 820, 920, 1020, 1120, 1220, 1320, 1420, and/or 1520, QDs 140, 640, and/or 740, and/or MND 670 and/or 770), regardless of composition, and may be advantageous even for small nanoparticles (e.g., nanoparticles less than 15 nm in size).

When adding a silica layer (e.g., silica coating 130, 630, and/or 730) to colloidal nanoparticles, it may be advantageous to control the thickness of the deposited silica. The thickness of the deposited silica may be between about 0.7 nm and about 7.7 nm using the S⁴C approach by altering the amount of silica precursor (e.g., tetraethyl orthosilicate, TEOS) (see FIGS. 7H-7I). In contrast, the standard RMM protocol was unable to achieve a similar level of control of the thickness of the deposited silica, as unevenly coated multi-core particles readily formed, especially for larger particles and/or thinner silica shells.

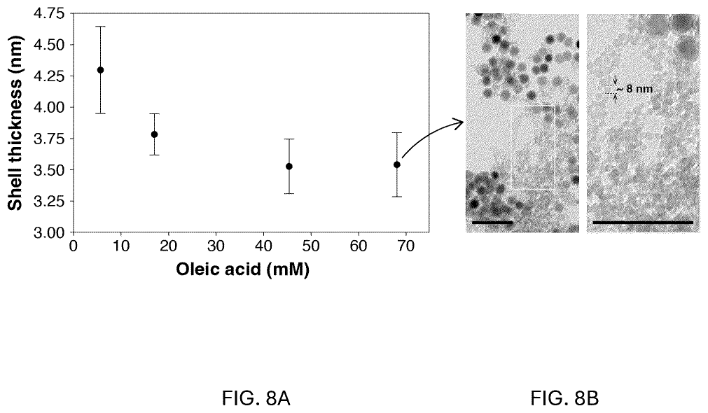

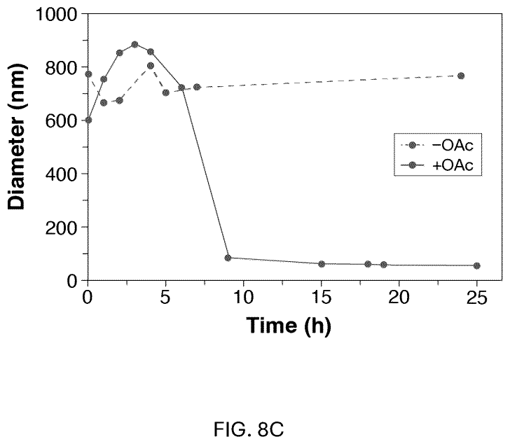

These results indicate that the initial surface ligand coating conformation may affect the quality of the silica coating 130, 630, and/or 730. To better understand the effect of oleic acid on the S⁴C approach, the concentration of OAc in the reaction solution was varied. As the oleic acid concentration increased from about 5 mM to about 45 mM, the shell thickness decreased (FIG. 8A). At concentrations above 45 mM, no further decrease in shell thickness was observed, but many silica nanospheres (with a thickness of approximately 8 nm) without core MNPs were formed (FIG. 8B). The presence of these “empty” silica spheres may suggest that oleic acid molecules can form small reverse micelles in the solution that are not sufficiently large or flexible to contain an MNP (e.g., MNPs 120, 220, 320, 420, 520, 620, 720, 820, 920, 1020, 1120, 1220, 1320, 1420, and/or 1520). Consequently, TEOS molecules may be hydrolyzed in the oleic acid reverse micelles, leading to the formation of core-free silica nanospheres. Since some TEOS molecules are consumed to create these core-free silica nanospheres, there may be less TEOS available to create silica shells on MNPs 120, 220, 320, 420, 520, 620, 720, 820, 920, 1020, 1120, 1220, 1320, 1420, and/or 1520, resulting in thinner shells. At concentrations below 45 mM, OAc may be mostly consumed by the particle surfaces and partially mixed with Igepal CO-520 to form reverse micelles together, and thus may not be present at high enough concentration to form oleic acid reverse micelles. While these data indicate that care should be taken in determining how much excess OAc to add in the S⁴C process, the smaller size and lower density of the silica nanospheres may enable them to be readily removed by centrifugation.

The role of oleic acid in enhancing the colloidal stability of core particles was also investigated by studying the hydrodynamic diameter of MNPs during the silica coating reaction using DLS (see FIG. 8C). MNPs aggregated immediately after the reaction solution was prepared. Without oleic acid, the diameter remained fairly constant, ranging between about 600 nm to about 800 nm even after 24 hours. In contrast, with oleic acid, the hydrodynamic diameter decreased to approximately 50 nm after 7 hours to 9 hours, even though initial aggregation behavior was similar to the non-oleic acid control. This finding indicates that the presence of oleic acid may improve the colloidal stability of MNPs 120, 220, 320, 420, 520, 620, 720, 820, 920, 1020, 1120, 1220, 1320, 1420, and/or 1520 over the course of the reaction, and thus may aid in preventing agglomeration or multi-core formation.

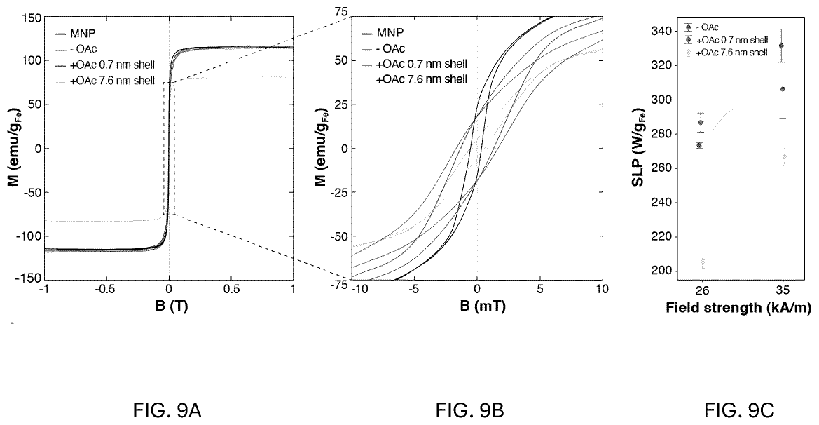

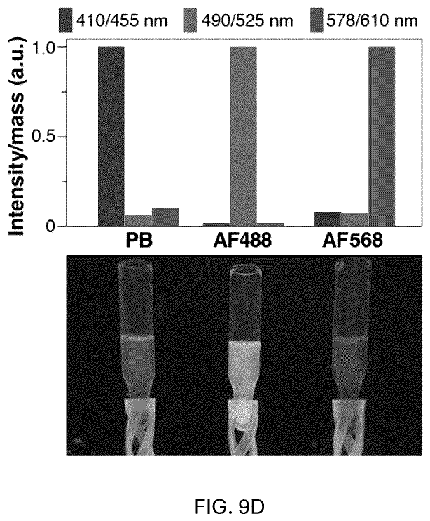

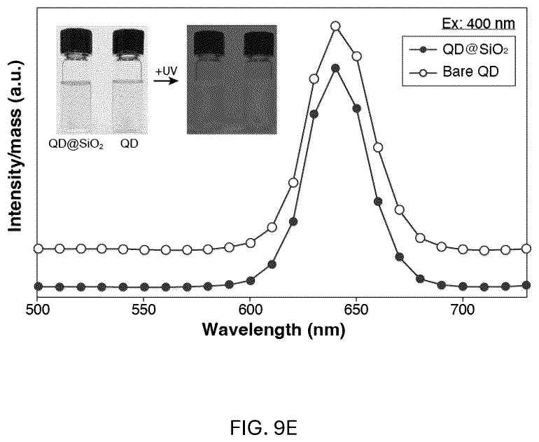

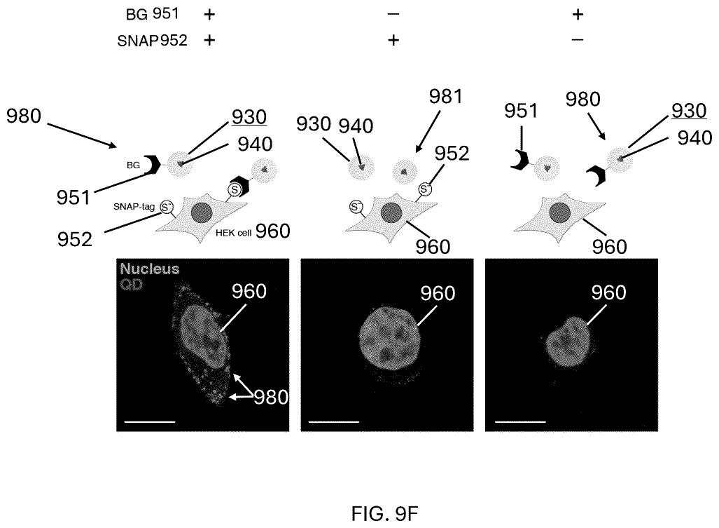

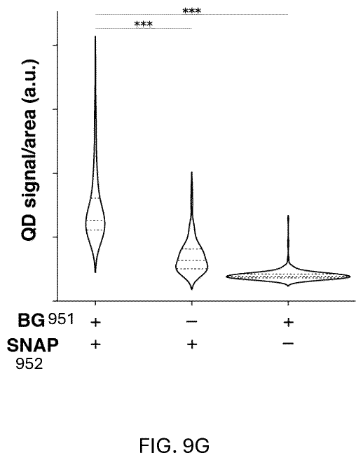

The S⁴C method may permit controlled silica shell growth on different nanoparticle compositions (e.g., MNPs 120, 220, 320, 420, 520, 620, 720, 820, 920, 1020, 1120, 1220, 1320, 1420, and/or 1520, QDs 140, 640, and/or 740, and/or MND 670 and/or 770). However, for these coated particles to remain useful in target applications, the properties of the cores should remain intact. For MNPs 120, 220, 320, 420, 520, 620, 720, 820, 920, 1020, 1120, 1220, 1320, 1420, and/or 1520, QDs 140, 640, and/or 740, and/or MND 670 and/or 770 disclosed herein, the saturation magnetization (Ms) may remain almost unchanged before and after the formation of silica shells 130, 630, and/or 730, except when the shell 130, 630, and/or 730 was particularly thick (e.g., about 7.6 nm or greater), where a decrease in Ms by approximately 27% was observed (FIGS. 9A and 9B). When 10 times more TEOS (e.g., about 200 μL) was added to the coating solution for MNDs 120, 220, 320, 420, 520, 620, 720, 820, 920, 1020, 1120, 1220, 1320, 1420, and/or 1520, the MNDs 120, 220, 320, 420, 520, 620, 720, 820, 920, 1020, 1120, 1220, 1320, 1420, and/or 1520 inside the silica shell 130, 630, and/or 730 decomposed. A similar chemical degradation of core MNPs 120, 220, 320, 420, 520, 620, 720, 820, 920, 1020, 1120, 1220, 1320, 1420, and/or 1520 may occur at the interface between MNPs 120, 220, 320, 420, 520, 620, 720, 820, 920, 1020, 1120, 1220, 1320, 1420, and/or 1520 and silica shells 130, 630, and/or 730 in the thick shell condition, causing the decrease in Ms. In the absence of oleic acid, the coercivity increased since multi-core particles develop uniaxial shape anisotropy, which may increase as more particles aggregate.

Specific loss power (SLP, W/g-Fe) was also measured for the same set of particles (FIG. 9C). The SLP of the thick shell particle decreased by approximately 19% compared to the oleic acid-free condition, which can be attributed to the smaller Ms. For the other two conditions, the SLPs were comparable, although the OAc-free condition had a slightly higher SLP. As vibrating-sample magnetometer (VSM) measurements illustrate, multi-core particles (−OAc) were magnetically harder than single-core particles (+OAc), leading to a larger hysteresis loop area. Since the area equates to energy loss in one cycle of magnetization rotation, the difference in SLP between-OAc and +OAc conditions is reasonable. In a smaller magnetic field, magnetically harder multi-core particles have a a higher field saturation threshold, and thus the SLPs of single-core particles are greater than that of multi-core particles.

For QDs 140, 640, and/or 740 coated with silica, the emission spectra were compared before and after the silica shell coating 130, 630, and/or 730 (FIG. 9E). The spectra were nearly identical with no noticeable degradation. Thus, silica shell formation by the S⁴C method may not impact the optical properties of QDs 140, 640, and/or 740.

Finally, one advantage of a uniform surface coating for nanoparticles (e.g., MNPs 120, 220, 320, 420, 520, 620, 720, 820, 920, 1020, 1120, 1220, 1320, 1420, and/or 1520, QDs 140, 640, and/or 740, and/or MND 670 and/or 770 (may be the ability to develop standardized functionalization protocols to alter their surface chemistry. To demonstrate that the S⁴C method enables such modification, silica shell coated particles (e.g., MNPs 120, 220, 320, 420, 520, 620, 720, 820, 920, 1020, 1120, 1220, 1320, 1420, and/or 1520, QDs 140, 640, and/or 740, and/or MND 670 and/or 770) were functionalized with primary amine groups by the addition of [3-(2-aminoethylamino) propyl]trimethoxysilane (AEAPTMS) via commonly used protocols developed for other silica nanoparticles. After purification, these amine-functionalized silica-coated nanoparticles were labeled with fluorescent dyes through NHS chemistry (FIG. 9D). Three different dyes, Pacific Blue (PB) (left column), Alexa Fluor 488 (AF488) (middle column), and Alexa Fluor 568 (AF568) (right column) were used, each of which possessed distinct excitation and emission profiles ((λₑₓ, λₑₘ)=(410, 455 nm), (490, 525 nm), and (578, 610 nm), respectively). Fluorescence measurements of these modified particles showed that each of these fluorophores was successfully attached to the particle surfaces, demonstrating that S⁴C produces silica-coated nanoparticles (e.g., MNPs 120, 220, 320, 420, 520, 620, 720, 820, 920, 1020, 1120, 1220, 1320, 1420, and/or 1520, QDs 140, 640, and/or 740, and/or MND 670 and/or 770) that may be directly inserted into the types of chemical modification protocols already in wide use by the scientific community.

To further demonstrate the utility of the S⁴C process in tailoring nanoparticle surface functionality, QDs 940 with a silica shell coating 930 were modified with moieties that permitted their targeting of HEK293T cells 960 (FIG. 9F). Specifically, SNAP-tag® technology—SNAP-tag 952, a protein which forms a covalent bond with O⁶-benzylguanine (BG) 951—was used. Thus, amine-functionalized silica-coated QDs were modified with both BG 951 and polyethylene glycol (PEG) (not shown) via NHS chemistry (QD-PEG/BG 980); PEG may improve the colloidal stability, which may be useful for minimizing non-specific interactions between QDs 940 and HEK cells 960. As a control group, QDs 940 modified with only PEG without BG were prepared (QD-PEG 981). Fluorescence imaging revealed a significant presence of QDs 940 on the membrane of HEK cells 960 that expressed SNAP-tags 952 and were cultured with QD-PEG/BG 980 (FIG. 9F, left panel). In contrast, control groups-SNAP-tag expressing HEK cells 960 treated with QD-PEG 981 (FIG. 9F, middle panel) and non-transfected HEK cells 960 incubated with QD-PEG/BG 980 (FIG. 9F, right panel) exhibited fewer QDs 940 on the cell membrane. When normalized by cell area, the fluorescence intensity from QDs 940 showed significant differences between the main group and the controls (FIG. 9G).

The S⁴C method disclosed herein, which incorporates oleic acid into the silica shell formation solution used in RMM, may improve the uniformity and reproducibility of the coating 130, 630, 730, and/or 930. The surface density of surfactant may also be a factor for achieving uniformly silica-coated particles. The addition of oleic acid may stabilize the nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770) even if the initial surface density of surfactant on the nanoparticles is not sufficiently high, leading to uniformly and individually coated particles. Moreover, the S⁴C method may improve the reproducibility of the coating 130, 630, 730, and/or 930. The poor reproducibility of RMM may be attributed to the variability of the surface density of oleic acid on nanoparticles. While the exact role of oleic acid in the S⁴C method remains to be elucidated, the results disclosed herein may reveal that nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770) may aggregate at the initial stage of the coating reaction and may imply that oleic acid may help disperse these clusters at an intermediate stage (FIG. 8C). With sufficient surfactant molecules on the core nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770), these clusters may not form. However, the S⁴C method may be “robust”. For example, the S⁴C method may not be sensitive to the oleic acid quantity on the surface of core nanoparticles and may allow for the formation of uniform, single-core silica-coated particles.

RMM has been widely utilized to coat hydrophobic nanoparticles with silica. However, it presents challenges in the reproducibility and uniformity of the coating. The methods disclosed herein demonstrate that the addition of oleic acid to the reaction solution for RMM may prevent the formation of multi-core particles and may also enhance the uniformity and reproducibility of silica shells (e.g., silica shells 130, 630, 730, and/or 930). The S⁴C method disclosed herein is versatile and may be suitable for relatively large (e.g., 20-28 nm) spherical MNPs 120, 220, 320, 420, 520, 620, 720, 820, 920, 1020, 1120, 1220, 1320, 1420, and/or 1520, even larger (e.g., 30×200 nm) non-spherical MNDs 670 and/or 770, and nanoparticles with varying compositions (e.g., quantum dots 140, 640, 740, and/or 840). Amine functionalization of silica shells 130, 630, 730, and/or 930, formed by the S⁴C method, may be achieved by adding an amine source material, e.g., AEAPTMS, to the same solution. The amine-functionalized silica shells may then be readily modified with variety of moieties through NHS chemistry in a precisely controlled fashion. The S⁴C method therefore may offer a straightforward and powerful way to create monodisperse silica layers with uniform thickness on various nanoparticles (e.g., e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770), and may serve as a universal platform for functionalization. For the S⁴C method, the nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770) may be initially coated with oleic acid. Furthermore, many synthetic protocols for high-quality nanoparticles are conducted in hydrophobic organic solvents and use oleic acid as a surfactant, indicating potentially broad utility of this approach. Silica-coated particles produced using the S⁴C method may stimulate new avenues in material research, due to the enhanced uniformity and reproducibility of the coating process.

### Structural and Magnetic Characterization

Transmission electron microscopy images and electron diffraction patterns of all coated/non-coated nanoparticles were obtained with an FEI Tecnai G2 Spirit TWIN TEM. Fiji was used for visualization. Dynamic light scattering measurements were performed with a Nicomp Nano DLS/ZLS systems. The concentration of nanoparticles was taken by using an Agilent 5100 Inductively Coupled Plasma-Optical Emission Spectrometer (ICP-OES). Room-temperature hysteresis loops were measured by a vibrating sample magnetometer (VSM, Digital Measurement Systems Model 880A).

Specific loss power (SLP) was measured in a similar method to previously-described. Briefly, 50 μL of sample solutions in deionized (DI) water (2 mg/mL, n=3) were placed in small glass vial together with an optical fiber temperature prove (Omega HHTFO-101). The tube was placed at the center of an electromagnetic coil that is driven by a custom-built circuit (SI). The air gap between the sample tube and the coil is approximately 4 cm. Alternating magnetic field was applied with a frequency f=163 kHz and amplitude H₀=40 kA/m. The field amplitude was measured using an inductive pick-up coil installed next to the main coil. As a control measurement, about 50 μL of DI water without MNPs 120, 220, 320, 420, 520, 620, 720, 820, 920, 1020, 1120, 1220, 1320, 1420, and/or 1520 was used and any temperature change was not observed.

### In Vitro Specific Targeting Test

12-mm round coverslips (Electron Microscopy Sciences, #72196-12, #1 thickness) were coated with Matrigel (Corning) in a 1:30 dilution by the standard thin coating method provided by the manufacturer and placed in a 24-well plate. HEK293T cells were seeded on the coverslips in 1 mL of Dulbecco's Modified Eagle Medium (DMEM, GlutaMAX supplement, Gibco) with 2.5% fetal bovine serum (FBS, Cytiva) and transfected when cells reached about 70% confluency by adding a mixture of 4 μL of Lipofectamine 2000 (Invitrogen) and 1 μg of sDNA plasmid (pAAV-(MV::SNAPtm) in 50 μL of Opti-MEM (Gibco). HEK cells were cultured at 37° C. with 5% CO₂. Media was exchanged for fresh media 6 hours after transfection. Then, 48 hours after transfection, 2 μg of either QD-PEG/BG or QD-PEG in DI water was added to the medium. After incubation with QDs for 15 minutes, the cells were washed with phosphate-buffered saline (PBS) 2 times and then fixed for 15 minutes in 4% paraformaldehyde in PBS. After three washes with PBS, cells were stained with BioTracker 488 (Sigma-Aldrich) in a 1:1000 dilution in PBS for 15 minutes. After three washes with PBS, coverslips were mounted onto glass slides using Fluoromount-G (Invitrogen).

Targeting specificity of QD-PEG/BG 980 was evaluated and quantified using a Leica DMI8 Inverted Confocal Microscope. For quantification, a 20× objective lens was used to obtain fluorescence images. For high magnification images, a 60× objective lens was used. QDs 140, 640, 740, and/or 840 were excited with a 400-nm diode laser and detected at about 620-660 nm. The images were quantified by using CellProfiler.

### Nanomagnetic Simulations

Nanomagnetic simulations were performed using MuMax3. Particles were about 24 nm spheres with edge to edge spacing of 2 nm (from TEM, FIG. 7B). The spheres had magnetic properties of magnetite: 110 emu/g-Fe (from VSM) and exchange constant=1.3×10⁻¹² J/m. Hysteresis measurements were performed by slightly varying the applied external field and allowing the simulation to relax to its lowest energy state.

Trans-Cyclooctene Functionalization of MNP@SiO₂—NH₂

MNP@SiO₂—NH₂ was functionalized with TCO groups through NHS chemistry using TCO-PEG24-NHS. For better stability in ionic solutions, mPEG24-NHS was mixed together. The grafting density of 1/nm² was assumed, and 20× equivalent polymers were used. The molar ratio was NHS-PEG24-TCO:NHS-mPEG24=30:70. Typically, about 2.38 mg of TCO-PEG24-NHS and about 4.38 mg of mPEG24-NHS were mixed in DMSO, then 1.6 mg-Fe of MNP@SiO₂—NH₂ (88 μmol) in DMSO was added. The concentration of MNP 120, 220, 320, 420, 520, 620, 720, 820, 920, 1020, 1120, 1220, 1320, 1420, and/or 1520 was about 129 nM. The solution was immediately set on a vortex and mixed for 24 hours at room temperature. The TCO-functionalized MNPs (MNP@SiO₂-TCO) (e.g., 120, 220, 320, 420, 520, 620, 720, 820, 920, 1020, 1120, 1220, 1320, 1420, and/or 1520) were purified by centrifuge at 20,000 g for 20 minutes at room temperature. The pellet was re-suspended in 1 mL of Milli-Q water and sonicated to disperse, then further washed with 1 mL of Milli-Q water on a MACS magnetic separation column (MS Columns #130-042-201, Miltenyi Biotec) three times. The washed MNP@SiO₂-TCO was eluted in 400 μl of Milli-Q water and stored at 4° C. The concentration was determined by ICP-AES.

### Functionalization of Amine-Functionalized Silica Shells Through NHS Chemistry

Amine-functionalized silica shells were modified with dyes as described above. The dyes may include but are not limited to: PB-NHS: Fluoroprobes #1245-5, AF488-NHS and AF568-NHS: Lumiprobe, #11820 and #14820, respectively, methoxy PEG (mPEG24-NHS; BroadPharm #BP-23970) and/or BG-PEG5k-NHS through NHS chemistry. BG-PEG5k-NHS was synthesized via NHS chemistry between BG-NH₂ (AmBeed #A455042) and NHS-PEG5k-NHS (Nanocs, #PG2-THTZ-5k). About 1 equivalent amount of NHS-PEG5k-PEG was mixed with an about 1.5 equivalent amount of BG-NH₂ in DMSO for 18 hours. For all types of silica-coated nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770), ligand density of 1 ligand/nm² was assumed and 20× equivalent of the number of ligands were used for NHS chemistry. The particles and ligands were mixed in DMSO on a vortex for 48 hours. The functionalized silica-coated nanoparticles (e.g., MNPs 120, 220, 320, 420, 520, 620, 720, 820, 920, 1020, 1120, 1220, 1320, 1420, and/or 1520, QDs 140, 640, 740, and/or 840, and/or MND 670 and/or 770) were purified in DI water by 3 rounds of centrifugation at 20 kg for 20 minutes and stored at 4° C.

### Adeno-Associated Viruses Packaging

All the adeno-associated viruses (AAVs) used in the experiments disclosed here were packaged in the lab through a previously established protocol. Briefly, a capsid plasmid (e.g., pAAV2/9n), a helper plasmid (pHelper), and a plasmid for the gene of interest (e.g., pAAV-CAG::NLS-GFP) were used for PEI-mediated triple transfection of HEK293T cells using PEI MAX® (Polysciences #24765-100). The culture medium was collected at 72 hours and 120 hours, and the cells were harvested at 120 hours post-transfection, followed by purification by ultracentrifugation. The AAVs were collected in Dulbecco's phosphate-buffered saline (DPBS) (Gibco) with 0.1% Pluronic F-68 (Gibco). AAV titers were determined using a Taraka Bio AAV real-time PCR titration kit (#6233). The AAVs were stored at 4° C. for up to 3 months.

### Chemical Modification of AAVs

AAVs 110, 210, 310, 410, 510, 610, 710, 810, 910, 1010, 1110, 1210, and/or 1310 were chemically modified with tetrazine (Tz) or fluorescent dyes through NHS chemistry with primary amine groups in the lysine residues on AAV capsids 110, 210, 310, 410, 510, 610, 710, 810, 910, 1010, 1110, 1210, and/or 1310. First, to adjust the pH of AAV solution, the buffer of AAV solution was exchanged with pH 8.4 sodium bicarbonate solution in Dulbecco's phosphate-buffered saline (DPBS) (Gibco; No Ca, no Mg) with 0.1% Pluronic F-68 (Gibco) using Amicon Ultra 0.5 mL Centrifugal Filters (#UFC510096, Millipore Sigma). The filter was pre-wetted with 400 μL of the buffer and spun at 3,500 g for 4 minutes. Then, 4 pmol of AAVs 110, 210, 310, 410, 510, 610, 710, 810, 910, 1010, 1110, 1210, and/or 1310 was loaded in one filter and the total volume was adjusted to 450 μL with the pH 8.4 DPBS-F68 buffer. The filter unit was spun at 3,500 g for 4 minutes. After the spin, the volume was typically about 120 μL. The pH 8.4 DPBS-F68 was added up to 450 μL and spun at the same condition. This buffer exchange centrifugation was repeated two more times (four times in total). After the 4th spin, the buffer-exchanged AAV solution was collected in a new tube by centrifugation.

Then, the volume of buffer-exchanged AAV solution was adjusted to 260 μL using the pH 8.4 DPBS-F68. 1 equivalent amount of NHS ligand to the accessible lysine residues was used for functionalization (Tz/Lys=1). For example, for AAV-DJ 610, it was assumed that there were 480 accessible lysine residues on its capsid (see Table 1). 7.78 μL of Tz-PEG4-NHS in DMSO (0.15 mg/mL) was added to the buffer-exchanged AAV solution. The solution was briefly vortexed and reacted at 4° C. for 24 hours. The AAV-Tz were purified with DPBS-F68 (pH 7.4) using Amicon® Ultra 0.5 mL centrifugal filters 6 times. After the purification, the volume of AAV-Tz was typically about 100 μL. The titer of AAV-Tz was determined using a Taraka Bio AAV real-time PCR titration kit (#6233).

This reaction was controlled based on the number of Tz per accessible lysine residues on an AAV capsid 110, 210, 310, 410, 510, 610, 710, 810, 910, 1010, 1110, 1210, and/or 1310. The number of Tz on an AAV capsid 110, 210, 310, 410, 510, 610, 710, 810, 910, 1010, 1110, 1210, and/or 1310 after the reaction was not measured, but instead functional tests of AAV-Tz's as viral vectors in vitro and in vivo were performed (FIGS. 12A-13).

### Chimerization of MNPs and AAVs

MNP@SiO₂-TCO and AAV-Tz were conjugated through the inverted electron-demand Diels Alder (IEDDA) reaction. mPEGSk-Tz was used as a quencher. Sulfo-Cy7-Tzwas also used as a quencher when MNPs 120, 220, 320, 420, 520, 620, 720, 820, 920, 1020, 1120, 1220, 1320, 1420, and/or 1520 were fluorescent-labeled for visualization. The procedure for the standard reaction condition is as follows: AAV-Tz (2 pmol; 60 μL at 2.0×10¹³ vg/mL) mPEG5k-Tz (10.33 μL at 0.5 mg/mL in DPBS-F68), and Cy7-Tz (4.8 μL at 0.25 mg/mL in DPBS-F68) were mixed with DPBS-F68 (128 μL). MNP@SiO₂-TCO (1.8 μL at 1.45 mg-Fe/mL) was diluted in DPBS-F68 (128 μL). The MNP@SiO₂-TCO solution was added to the AAV-Tz+quenchers solution on a vortex. After mixed, the solution was vortexed for 10 seconds and reacted for 30 minutes at room temperature. Then, extra mPEGSk-Tz (12.4 μL at 0.5 mg/mL in DPBS-F68) was added on a vortex to completely inactivate the TCO groups on MNPs 120, 220, 320, 420, 520, 620, 720, 820, 920, 1020, 1120, 1220, 1320, 1420, and/or 1520 and reacted at 4° C. overnight.

The synthesized MNP-AAV chimeras 100, 200, 300, 400, 500, 600, 700, 800, 900, 1000, 1100, 1200, and/or 1300 were purified by centrifugation at 15,000 g for 10 minutes at 4° C. with 200 μL of 0.01 g/L of mPEG4-TCO in DPBS-F68. The purification was repeated four times to fully remove unreacted free AAV-Tz, and the pellet was resuspended in 100 μL of DPBS-F68 and stored at 4° C.

### Characterization of MNP and MNP-AAV Chimeras

The AAV/MNP ratio was determined from TEM images. More than 600 entities were counted and quantified. The places to image were chosen randomly to remove operator's bias. Images were taken using a FEI Tecnai G2 Spirit TWIN TEM at the acceleration voltage of 120 kV.

### In Vitro Targeting Specificity Test

HEK293T cells, HeLa cells, and C2C12 cells were used in in vitro targeting specificity tests. HEK293T, HeLa, and C2C12 cells were cultured in Dulbecco's Modified Eagle Medium (DMEM, Gibco #10569044) with 10% of fetal bovine serum (FBS, Cytovia #SH30396.03HI) and passaged at 90% confluency.

In AAV serotype specificity test, the cells were seeded in a 6-well plate (cell culture treated). At 24 hours post seeding (approximately 70% confluency), AAVs 110, 210, 310, 410, 510, 610, 710, 810, 910, 1010, 1110, 1210, and/or 1310 that packaged pAAV-CAG::mRuby2 were added to the medium and incubated for 24 hours. The mRuby2 fluorescence was measured by flow cytometry (BD FACSCelesta). The gating conditions can be found in FIG. 22.

In the targeting specificity test of MNP-AAV chimeras 100, 200, 300, 400, 500, 600, 700, 800, 900, 1000, 1100, 1200, and/or 1300, the cells were seeded in a glass-bottom 96-well plate coated with Matrigel for 1 hour (Corning #356234). At 24 hours post seeding (approximately 70% confluency), cells were stained with CellMask™ Deep Red (Invitrogen C10046, 1:1000 dilution) and NucSpot Live 488 (biotium #40081, 1:1000 dilution) for 10 minutes. The cells were washed with DMEM twice, then chimeras 100, 200, 300, 400, 500, 600, 700, 800, 900, 1000, 1100, 1200, and/or 1300 were added (0.3 μg-Fe/well). After 1 hour of incubation, cells were washed with phosphate-buffered saline (PBS) twice and fixed with 4% paraformaldehyde in PBS for 15 minutes. The cells were washed with PBS three times and imaged on a confocal microscope (Leica Stellaris 5) with a 20× objective lens. For determining the percentages of Cy7 positive cells, AAV9 chimera 800 and/or 1100 was used as a “non-targeting” condition, and the threshold was defined at the 95% of Cy7 fluorescence intensity of AAV9 chimera 800 and/or 1100. For the evaluation of the correlation coefficient between mRuby2+% and MNP-Cy7+%, the pearson function in the scipy module was employed on Python.

Quantification of Fluorescence Intensity from MNP-Cy7

In vitro fluorescence images were quantified using Cell Profiler. The mean Cy7 intensity in each cell, I_{cell,i}, was calculated by Equation 2:

$${I}_{ceil , i} = \frac{{Σ}_{j}^{n} {I}_{p i xel , j}}{n} , \qquad ( 2 )$$

where n is the number of pixels in each cell.

An average of I_{cell,i} in a sample was calculated by Equation 3:

$${I}_{s a m p l e} = \frac{{Σ}_{i}^{N} {I}_{c ell , i}}{N} , \qquad ( 3 )$$

where N is the number of cells in the sample.

### Magnetic Guidance of MNP-AAV Chimeras for Spatially Restricted Transduction

HEK cells were seeded in a 35 mm dish with a 20-mm coverslip window (Mattek #P35G-1.5-20-C) coated with Matrigel for 1 hour. At 24 hours post seeding (approximately 70% confluency), a cone-shape magnet was placed right under the center of the coverslip, then DJ chimera 600 was added to the solution. The dish with a magnet was swirled/shaken to mix and incubated. At 1 minute, the medium was completely removed, and a fresh medium was added (e.g., about 2 mL). At 24 hours post transduction, cells were washed with PBS twice and fixed with 4% PFA in PBS for 15 minutes. After 3 cycles of washing with PBS, the cell nucleus was stained with DAPI (1:20,000) for 15 minutes. Cells were washed with PBS twice and imaged on a confocal microscope (Leica Stellaris 5) with a 10× objective lens.

The fluorescence intensity of GFP was quantified using custom Python code. Briefly, the intensity was normalized by summing the fluorescence intensities of pixels equidistant from the magnetic center and dividing by the total area of the pixels involved in the summation. This process was conducted in 30 μm steps. Moving averages (data points=10) were also plotted.

Finite element method magnetic field gradient simulation was performed using the AC/DC module on COMSOL Multiphysics. The dimensions of the cone magnet on the simulation were the same size as the actual magnet used in the experiment. As a material, the NdFeB in the COMSOL's standard data set was used.

### In Vivo Delivery Test

4-week-old C57BL/6 mice were purchased from the Jackson Laboratory. Mice were fed with an alfalfa-free diet (LabDiet AlN-93M) for two weeks prior to imaging experiments. The furs were removed one day before the imaging test. In vivo fluorescence images were obtained before injection and at 2 hours, 4 hours, 12 hours, and 24 hours post injection using IVIS Spectrum with a 745 nm excitation filter and an 800 nm emission filter (Perkin Elmer). At desired time points (24 hours, 48 hours, and 2 weeks), 100 μL of Tomato Lectin DyLight 488 (Vector laboratories #DL-1174-1) was injected to the mice via the retro-orbital route. After 10 minutes, the mice were perfused with PBS and 4% PFA. The brain and liver were dissected and post-fixed in 4% PFA for 48 hours and subsequently kept in PBS for another 48 hours to remove residual PFA. The tissue samples were imaged with IVIS Spectrum to obtain ex vivo fluorescence images. For confocal imaging of tissue samples, LEICA VT1000 vibraotme was used to slice the tissues at 50 μm (brains) and 60 μm (livers). Slice samples were washed with PBS for 10 minutes on a shaker three times, then stained with DAPI (1:20,000) for 20 minutes. After 2 more washing with PBS, the slices were mounted on a slide glass with Fluolomount-G (Invitrogen, 00-4958-02).

### Fluorescence Image Quantification

Fluorescence images (in vivo, ex vivo) were quantified using Living Image (PerkinElmer). In FIG. 5C, the initial radiant efficiency measured at t=0 hours was set to zero. In FIG. 5D, raw data was plotted without any preprocessing. In FIG. 5E, the brain delivery efficiency was evaluated by Equations 4-6:

$${I}_{brain , backgorund \_ corrected} = {I}_{B r a i n , r a w} - {I}_{P B S} ; \qquad ( 4 )$$

$${m}_{F e , brain} = \frac{{1}_{brain , background \_ corrected} + 4.26 \times 1 {0}^{6}}{7.49 \times 1 {0}^{7}} ; \qquad ( 5 )$$

$$Delivery efficiency = \frac{{m}_{Fe , brain}}{{m}_{injected}} . \qquad ( 6 )$$

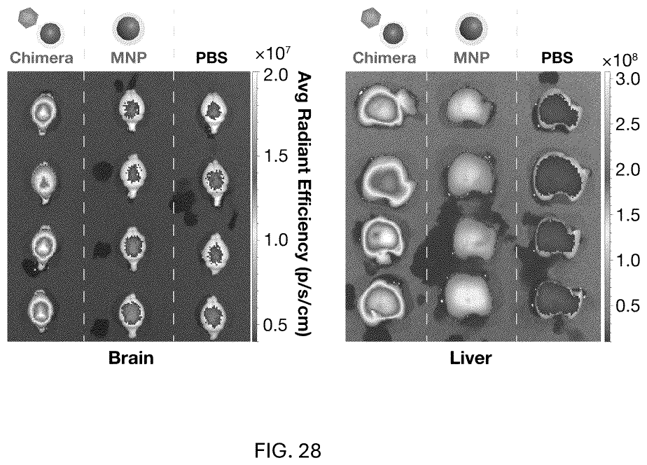

In Equation 3, the mean fluorescence in the PBS control (I_{PBS}) was considered as a background and subtracted from the intensity in the brain (I_{Brain,raw}). Bayesian estimation was employed to calculate I_{PBS} from the measurement data (N=4). The Markov Chain Monte Carlo sampling was used to estimate the posterior distributions and 10000 sampling iterations were performed, and I_{PBS} was computed as the mean of the 10000 sampled values. Equation 4 was used for converting fluorescence intensity to the mass of Fe (m_{Fe,brain}) from a standard curve measurement (FIG. 28). To make the standard curve, a dilution series of MNP-Cy7 mixed with 1% agarose gel was prepared. The fluorescence intensity of those gels was measured using the same IVIS machine and settings used for in vivo and ex vivo IVIS imaging of animals and tissues. The delivery efficiency was calculated by dividing the mass of Fe in the brain by the mass of Fe of the total injected dose (m_{injected}). For calculating % ID/g brain from % ID, the weight of the brain was assumed to be 0.4 g based on Jax's data.

For the liver-targeting AAV9 chimeras 800 and/or 1100, the fluorescence intensity of the liver was background-subtracted using the PBS control data, then normalized by the amount of MNPs 820 and/or 1120 (g-Fe) injected.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the components so conjoined, i.e., components that are conjunctively present in some cases and disjunctively present in other cases. Multiple components listed with “and/or” should be construed in the same fashion, i.e., “one or more” of the components so conjoined. Other components may optionally be present other than the components specifically identified by the “and/or” clause, whether related or unrelated to those components specifically identified. Thus, as a non-limiting example, a reference to “A and/or B”, when used in conjunction with open-ended language such as “comprising” can refer, in one embodiment, to A only (optionally including components other than B); in another embodiment, to B only (optionally including components other than A); in yet another embodiment, to both A and B (optionally including other components); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of components, and, optionally, additional unlisted items. Only terms clearly indicated to the contrary, such as “only one of” or “exactly one of,” or, when used in the claims, “consisting of,” will refer to the inclusion of exactly one component of a number or list of components. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more components, should be understood to mean at least one component selected from any one or more of the components in the list of components, but not necessarily including at least one of each and every component specifically listed within the list of components and not excluding any combinations of components in the list of components. This definition also allows that components may optionally be present other than the components specifically identified within the list of components to which the phrase “at least one” refers, whether related or unrelated to those components specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including components other than B); in another embodiment, to at least one, optionally including more than one, B, with no A present (and optionally including components other than A); in yet another embodiment, to at least one, optionally including more than one, A, and at least one, optionally including more than one, B (and optionally including other components); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A gene-targeting chimera comprising:
at least one adeno-associated virus (AAV);
a nanoparticle;
a uniform silica shell coating over the nanoparticle; and
a linking chemistry covalently attaching the at least one AAV and the nanoparticle.

2. The gene-targeting chimera of claim 1, wherein the at least one AAV is up to eight AAVs.

3. The gene-targeting chimera of claim 2, wherein the at least one AAV consists of one AAV.

4. The gene-targeting chimera of claim 1, wherein the nanoparticle comprises at least one of a magnetic nanoparticle (MNP), a magnetic nanodisc (MND), or a quantum dot (QD).

5. The gene-targeting chimera of claim 4, wherein the nanoparticle is the MNP.

6. The gene-targeting chimera of claim 5, wherein the MNP is about 20 nm to about 25 nm in diameter.

7. The gene-targeting chimera of claim 5, wherein the MNP comprises a spherical MNP or a faceted MNP.

8. The gene-targeting chimera of claim 1, wherein the linking chemistry comprises a click chemistry reaction.

9. The gene-targeting chimera of claim 8, wherein the click chemistry reaction is an inverse electron-demand dials-alder (IEDDA) reaction.

10. The gene-targeting chimera of claim 1, wherein the uniform silica shell coating is about 2 nm to about 8 nm thick.

11. The gene-targeting chimera of claim 1, wherein a location of the gene-targeting chimera in a mammal may be controlled by at least one of a serotype of the AAV or a magnetic field.

12. The gene-targeting chimera of claim 1, wherein the gene-targeting chimera is contained in a solution comprising a plurality of AAVs and nanoparticles wherein each AAV is covalently attached to the nanoparticle.

13. A method comprising:
coating a nanoparticle with a uniform shell of silica;
covalently attaching the nanoparticle to at least one adeno-associated virus (AAV) to form a gene-targeting chimera, wherein the at least one AAV is up to eight AAVs; and
intravenously injecting the gene-targeting chimera into a mammal.

14. The method of claim 13, further comprising controlling a location of the gene-targeting chimera using at least one of a magnetic field or a serotype of the AAV within the mammal.

15. The method of claim 13, wherein the covalently attaching the nanoparticle to the AAV further comprises controlling a ratio of the at least one AAV to the nanoparticle using a quenching agent.

16. The method of claim 15, wherein the quenching agent comprises tetrazine-methoxy polyethylene glycol or trans-cyclooctene methoxy polyethylene glycol.

17. The method of claim 13, wherein the covalently attaching the nanoparticle to the AAV comprises a click chemistry reaction.

18. The method of claim 17, wherein the click chemistry reaction comprises an inverse electron-demand dials-alder (IEDDA) reaction.

19. The method of claim 13, wherein the coating the nanoparticle with silica comprises adding oleic acid to control a thickness of the uniform shell of silica.

20. The method of claim 13, wherein the nanoparticle comprises at least one of a magnetic nanoparticle (MNP), a magnetic nanodisc (MND), or a quantum dot (QD).
